(19) United States
(12) Patent Application Publication
(10) Pub. No.: US 2026/0345857 A1
(43) Pub. Date: Nov. 5, 2026

(54) MULTIFUNCTIONAL STRUCTURES AND LAMINATES

(71) Applicant: DelStar Technologies, Inc., Alpharetta, GA (US)

(72) Inventor: Nandan Ukidwe, Suffield, CT (US)

(21) Appl. No.: 19/655,932

(22) Filed: Apr. 23, 2026

Related U.S. Application Data
(60) Provisional application No. 63/793,887, filed on Apr. 24, 2025.

(51) Int. Cl. A61L 15/26; A61L 15/20; A61L 15/42; B32B 7/06; B32B 7/12; B32B 27/18; B32B 27/40

(52) U.S. Cl. CPC A61L 15/26; A61L 15/20; A61L 15/42; B32B 7/06; B32B 7/12; B32B 27/18; B32B 27/40; B32B 2307/412; B32B 2307/7376; B32B 2535/00

(57) ABSTRACT

Compositions, composites, laminates, bandages, dressings and/or films include one or more optical agents, materials, and/or layers capable of blocking ultraviolet (UV) radiation while being substantially transparent to visible light. An interlayer or layer includes a thermoplastic polyurethane and a UV blocker disposed in the thermoplastic polyurethane layer. The UV blocker includes a material in the imidazole family, a material in the cyanoacetate family or a combination thereof. The interlayer may, for example, be disposed between first and second optically transparent sheets to form a laminate or composite for a window. The UV blockers in the interlayer block greater than or equal to about 98% of light having a wavelength of about 400 nm, while mitigating certain risks, such as carcinogenic, mutagenic and reproductive toxicity risks, that could otherwise raise environmental or health concerns.

### CROSS-REFERENCE TO RELATED APPLICATIONS

This application claims the benefit of U.S. Provisional Application Ser. No. 63/793,887, filed Apr. 24, 2025, the complete disclosure of which is incorporated herein by reference for all purposes.

### TECHNICAL FIELD

This description generally relates to compositions, composites, structures, laminates, bandages, dressings and/or films having one or more optical agents, materials, and/or layers capable of blocking ultraviolet (UV) radiation while being substantially transparent to visible light.

### BACKGROUND

Solar radiation includes electromagnetic energy spanning a range of wavelengths, which may be broadly categorized as ultraviolet (UV), visible, and infrared (IR) radiation. Visible light generally includes wavelengths from about 400 nm to about 700 nm, IR radiation extends from about 700 nm to about 1 mm, and UV radiation spans wavelengths from about 10 nm to about 400 nm. Although limited exposure to UV radiation may provide certain benefits, excessive or prolonged exposure can damage human skin and contribute to both acute and chronic health conditions.

Solar radiation transmitted through architectural elements such as windows provides desirable natural illumination in the visible spectrum, but also permits transmission of UV radiation. To reduce such undesirable UV transmission, conventional laminated glass structures typically incorporate functional coatings, interlayers, and/or optical filtering materials configured to attenuate UV radiation. While such approaches have been generally effective, ongoing efforts seek to further improve UV attenuation performance and material compositions.

### SUMMARY

This following is intended merely to introduce a simplified summary of some aspects of one or more implementations of the subject matter discussed herein. Further areas of applicability of the subject matter will become apparent from the detailed description provided hereinafter. This summary is not an extensive overview, nor is it intended to identify key or critical elements of the present teachings, nor to delineate the scope of the subject matter. Rather, its purpose is merely to present one or more concepts in simplified form as a prelude to the detailed description below.

Provided herein are thermoplastic materials, including layers (e.g., outer layers and/or interlayers), films, bandages, dressings, and composite structures, that incorporate one or more functional elements, such as UV-absorbing agents. Also described are laminates and systems (e.g., laminated glazing units) including such thermoplastic materials, as well as methods of making and using the same.

The disclosed materials and structures may be employed in a wide range of applications, including glazing for vehicles and architectural structures, impact-resistant articles (e.g., bullet-resistant glass), and decorative or functional films for windows, walls, or doors (e.g., tinting, colored, or reflective films). In certain embodiments, the thermoplastic layers and films incorporating the functional elements are particularly suitable for use in applications requiring control of solar radiation, including absorption and/or reflection of heat, infrared (IR), and/or ultraviolet (UV) radiation. Such materials may also be utilized in energy-related applications, including photovoltaic devices (e.g., solar cells), or combinations thereof.

In other embodiments, bandages or dressings are described that incorporate a thermoplastic layer and UV agents. The UV agents block the transmission of UV light to the patient's skin. UV light can impair the body's natural healing processes and weaken the skin's defense mechanisms, making it easier for bacteria and other pathogens to enter a wound or other skin disorder. The bandages and dressings may include, but are not limited to, an elastic or adhesive bandage, dressing, wound dressing, compression bandage, surgical dressing, Tulle dressing, alginate dressing, foam dressing, hydrogel dressing, hydrocolloid dressing, gauze roll, gauze pad and the like

In one aspect, an optical film includes one or more thermoplastic polyurethane resins, a first UV absorber and a second UV absorber. The first UV absorber is selected from the group consisting of the benzotriazole UV absorbers and triazine UV absorbers and second UV absorber is selected from the group consisting of imidazole UV absorbers and cyanoacetate UV absorbers.

The UV blocker(s) in the interlayer surprisingly and expectedly block greater than or equal to about 98% of light having wavelengths of about 380 nm to about 400 nm, while mitigating certain risks, such as carcinogenic, mutagenic and reproductive toxicity risks, that could otherwise raise environmental or health concerns. In an exemplary embodiment, the combination of UV blockers exclude, are free, or are substantially free of, one or more materials and substances identified by the Registration, Evaluation, Authorization, and Restriction of Chemicals (REACH), a system designed to manage and control the potential risks associated with hazardous chemicals.

In another aspect, a laminate includes a first optically transparent sheet, a second optically transparent sheet and an interlayer interposed between the first and second optically transparent sheets. The interlayer includes a thermoplastic polyurethane and a UV blocker disposed in the thermoplastic polyurethane layer. The UV blocker is selected from the group consisting of imidazole UV absorbers and cyanoacetate UV absorbers.

In another aspect, a window is provided including the laminate of any of the above embodiments.

In another aspect, a bandage includes a layer of thermoplastic polyurethane and a UV blocker disposed in the thermoplastic polyurethane layer. The UV blocker is selected from the group consisting of imidazole UV absorbers and cyanoacetate UV absorbers

The bandage may include, but is not limited to, an elastic or adhesive bandage, dressing, wound dressing, compression bandage, surgical dressing, Tulle dressing, alginate dressing, foam dressing, hydrogel dressing, hydrocolloid dressing, gauze roll, gauze pad and the like

Further areas of applicability of the subject matter will become apparent from the detailed description provided hereinafter. It should be understood that the detailed description and specific examples, while indicating some typical aspects of the subject matter, are intended for purposes of illustration only and are not intended to limit the scope thereof.

The recitation herein of desirable objects which may be met by various embodiments of the present description is not meant to imply or suggest that any or all of these objects may be present as essential features, either individually or collectively, in the most general embodiment of the present description or any of its more specific embodiments.

### BRIEF DESCRIPTION OF THE DRAWINGS

The accompanying drawings, which are incorporated in and constitute a part of this specification, illustrate several embodiments of the disclosure and together with the description serve to explain the principles of the disclosure.

FIG. 1 is a cross-sectional view of an exemplary laminate including a thermoplastic interlayer.

FIG. 2 is a cross-sectional view of an exemplary laminate including a thermoplastic interlayer.

FIG. 3 is a schematic cross-sectional view of a bandage including a thermoplastic material.

FIG. 4 is a schematic cross-sectional view of the bandage disposed adjacent a site of treatment.

### DETAILED DESCRIPTION

This description and the accompanying drawings illustrate exemplary embodiments and should not be taken as limiting, with the claims defining the scope of the present description, including equivalents. Various mechanical, compositional, structural, and operational changes may be made without departing from the scope of this description and the claims, including equivalents. In some instances, well-known structures and techniques have not been shown or described in detail so as not to obscure the description. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Except as otherwise noted, any quantitative values are approximate whether the word “about” or “approximately” or the like are stated or not. The materials, methods, and examples described herein are illustrative only and not intended to be limiting.

As used throughout, ranges are used as shorthand for describing each and every value that is within the range. It should be appreciated and understood that the description in a range format is merely for convenience and brevity and should not be construed as an inflexible limitation on the scope of any embodiments or implementations discussed herein. Accordingly, the range should be construed to have specifically included all the possible subranges as well as individual numerical values within that range. As such, any value within the range may be selected as the terminus of the range. For example, description of a range such as from 1 to 5 should be considered to have specifically included subranges such as from 1.5 to 3, from 1 to 4.5, from 2 to 5, from 3.1 to 5, etc., as well as individual numbers within that range, for example, 1, 2, 3, 3.2, 4, 5, etc. This applies regardless of the breadth of the range.

Additionally, all numerical values are “about” or “approximately” the indicated value, and consider experimental error and variations that would be expected by a person having ordinary skill in the art. It should be appreciated that all numerical values and ranges discussed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is discussed herein, any numerical value falling within the range is also specifically included.

As used herein, the expression “excludes” or is “free” of a material or substance may refer to a composition, component, or phase where the material is present in an amount of less than 1.0 wt %, less than 0.1 wt %, less than 0.05 wt %, less than 0.01 wt %, less than 0.005 wt %, or less than 0.0001 wt %, based on a total weight of the composition, component, or phase. As used herein, the expression “substantially free” of a material or substance may refer to a composition, component, or phase where the material is present in an amount of from about 1.0 wt % or more to less than 20.0 wt %, less than 10.0 wt %, less than 5.0 wt %, less than 3.0 wt %, less than 2.0 wt %, less than 1.8 wt %, or less than 1.5 wt %.

All references cited herein are hereby incorporated by reference in their entireties. In the event of a conflict in a definition with a cited reference, the present teachings control.

Thermoplastic layers (e.g., outer layers and/or interlayers), films, and composites containing functional elements (e.g., UV agents), and methods for the same are described. Laminates and systems (e.g., laminated glazing units) incorporating the thermoplastic layers, films, and composites, and methods for the same are also described. The laminates, films, and composites, and the thermoplastic layers thereof, may be used in a variety of applications, such as windows for vehicles and buildings, impact resistance devices, such as bulletproof glass, decorative films for windows, walls or doors, window tinting, colored or mirrored glass, window films, or the like. The thermoplastic layers and thermoplastic films containing the functional elements may be particularly useful in windows that absorb and/or reflect heat, IR, and/or UV light, photovoltaic devices (e.g., solar cells), or the like, or a combination thereof.

As used herein, the term or expression “thermoplastic interlayer”, “thermoplastic film”, “thermoplastic layer”, or the like may be used interchangeably, and may only be distinguished from one another by their application, use, dimensions, and/or location. For example, the expression “thermoplastic layer” may broadly refer to or include: an interlayer disposed between at least two layers (e.g., ridged plies), an outer layer, an independent layer or film, or a combination thereof. In another example, a thermoplastic interlayer may have the same properties and/or dimensions as a thermoplastic film and a thermoplastic layer; however, the thermoplastic interlayer is disposed between two rigid sheets, while the thermoplastic film or the thermoplastic layer may be disposed in varying locations. As such, features and/or properties described with respect to a thermoplastic interlayer may be applicable to a thermoplastic film or a thermoplastic layer and vice versa. Illustrative systems that may utilize, incorporate, or otherwise include the thermoplastic interlayers and/or films containing the functional elements may be or include, but are not limited to, luminescent concentrators (LCs), laminated glazing units (LGUs), windows for vehicles and buildings, impact resistance devices (e.g., bulletproof glass), decorative films for windows, walls or doors, window tinting, colored or mirrored glass, window films, or the like, or any combination thereof.

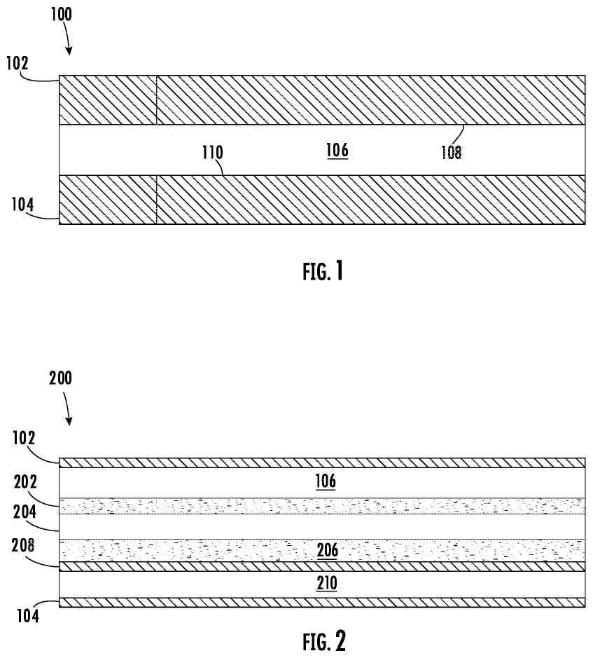

FIG. 1 illustrates a schematic cross-sectional view of a laminate 100, according to one or more implementations. As illustrated in FIG. 1, the laminate 100 may include first and second outer sheets or layers 102, 104 and a structure, such as a thermoplastic interlayer 106, interposed between the first and second outer layers 102, 104. The thermoplastic interlayer 106 may be a monolithic structure including one or more thermoplastic polymers (e.g., thermoplastic polyurethane) having a thickness sufficient to adhere the outer layers 102, 104 with one another. As used herein, the term or expression “monolithic” may refer to a single, substantially indivisible layer or film of material(s). It should be appreciated that the monolithic interlayer disclosed herein may include one or more layers or regions of materials that are substantially indivisible or substantially inseparable. As further described herein, the laminate 100 and/or the thermoplastic interlayer 106 thereof may be capable of or configured to selectively absorb, reflect, and/or transmit electromagnetic radiation (e.g., light) of varying wavelengths.

The outer sheets or layers 102, 104 may each include optically transparent and/or substantially rigid materials. Illustrative materials for the outer layers 102, 104 may be or include, but are not limited to, one or more of glass, artificial glass, or any well-known glass substitutes, such as polycarbonates, acrylic resins, polyesters, polyethers, laminated glass, polymeric sheets, ceramics, polyethylene terephthalates (PET), polyphosphonates, polyurethanes, a float, tempered, or chemically strengthened glass (e.g., borosilicate glass), or the like, or any combination thereof. In an exemplary implementation, the laminate 100 utilizes glass. Glass may provide a flat and abrasion resistant surface that may facilitate the fabrication or manufacture of the laminate 100 or a laminated glass incorporating the laminate 100. For example, the manufacturing process utilized to prepare laminated glass (e.g., safety glass) may be utilized to produce the laminate 100. Additionally, glass may exhibit relatively less absorption in the infrared (IR) region than polymers due, at least in part, to the absence of carbon-hydrogen bonds that have molecular vibration modes that may be excited in the wavelengths of from about 900 nm to about 1,000 nm.

In at least one implementation, interfaces 108, 110 between respective surfaces of the first outer layer 102 and the second outer layer 104 and the thermoplastic interlayer 106 may be reflective or non-reflective. Each of the interfaces 108, 110 may be independently reflective or non-reflective. The interfaces 108, 110 may be reflective to wavelengths of light or electromagnetic radiation from one or more of the visible, infrared, and/or ultraviolet regions of the spectrum, or subregions thereof.

The thermoplastic interlayer 106 may be extruded and assembled into the laminate 100. The thermoplastic interlayer 106 may be extruded by a single or two/double screw extruder, coated, cast, UV cured, or formed by other methods. In an exemplary implementation, the thermoplastic interlayer 106, including the functional elements (e.g., UV agents), may be extruded. Extrusion may include combining or otherwise contacting the one or more thermoplastic polymers (e.g., a thermoplastic polyurethane) and the functional elements (e.g., UV agents) with one another in a screw extruder, dispersing the functional elements in the thermoplastic polymers via the extruder, and extruding the thermoplastic polymers with the functional elements through a die to form the thermoplastic interlayer 106 in the form of a flat film, sheet, or layer. It should be appreciated that the ability to utilize extrusion casting with the thermoplastic interlayer 106 significantly reduces the cost of preparing the laminate 100 while concurrently providing the thermoplastic interlayer 106 with reduced haze (i.e., <5%) and/or improved visible light transmission.

The thermoplastic interlayer 106 may include one or more thermoplastic polymers. For example, the thermoplastic interlayer 106 may be or include a single thermoplastic polymer. In another example, the thermoplastic interlayer 106 may be or include a plurality of thermoplastic polymers. The plurality of thermoplastic polymers may be a homogenous or heterogenous mixture or blend thereof. The plurality of thermoplastic polymers may be a single-phase blend or a multi-phase blend. A single-phase blend of thermoplastic polymers may include a plurality of thermoplastic polymers that are miscible with one another and/or form a polymeric material, often with a single glass transition temperature (Tg) based on the ratio of the thermoplastic polymers. A multi-phase blend may include two or more thermoplastic polymers that may be immiscible with one another and/or have a plurality of glass transition (Tg) temperatures. As used herein, the term or expression “thermoplastic polymer” may refer to a plastic capable of being repeatedly softened or melted by increases in temperature and hardened by decreases in temperature. The one or more thermoplastic polymers may be crystalline or semi-crystalline. A crystalline thermoplastic polymer may generally include no crosslinking to a modest amount of crosslinking relative to a thermoset polymer. The relative decreased amount of crosslinking has a correspondingly reduced influence on respective properties of the thermoplastic polymer, such as softening and/or melting. A thermoplastic polymer may also generally include a higher molecular weight polymer relative to a thermoset, as a relatively higher molecular weight may enhance one or more properties, such as melting point to enhance or facilitate the integrity of the thermoplastic during use.

The thermoplastic polymer may be present in the thermoplastic interlayer 106 in an amount of from greater than or equal to about 80 wt % to about 99.99 wt %, based on the total weight of the thermoplastic interlayer 106. For example, the thermoplastic polymer may be present in an amount of from greater than or equal to about 80 wt %, greater than or equal to about 85 wt %, greater than or equal to about 90 wt %, greater than or equal to about 95 wt %, greater than or equal to about 98 wt %, greater than or equal to about 98.5 wt %, greater than or equal to about 99 wt %, greater than or equal to about 99.5 wt %, greater than or equal to about 99.9 wt %, or about 99.99 wt %. In an exemplary implementation, the thermoplastic interlayer 106 includes the thermoplastic polymer in an amount of from about 98 wt % to about 99.9 wt %, or about 99 wt %, based on the total weight of the thermoplastic interlayer 106.

The one or more thermoplastic polymers may have a molecular weight of from about 1,000 Da to about 250,000 Da or more. For example, any one or more of the thermoplastic polymers may have a molecular weight of from about 1,000 Da to about 250,000 Da, about 1,000 Da to about 200,000 Da, about 1,000 Da to about 150,000 Da, or about 1,000 Da to about 100,000 Da. In another example, any one or more of the thermoplastic polymers may have a molecular weight of from about 1,000 Da, about 2,000 Da, about 5,000 Da, about 10,000 Da, about 50,000 Da, or more to about 100,000 Da, about 150,000 Da, about 200,000 Da, about 250,000 Da, about 300,000 Da, about 350,000 Da, or more.

In an exemplary implementation, the thermoplastic polymer may be or include one or more thermoplastic elastomers. An elastomer may include a macromolecular material that may return rapidly to approximately the initial dimensions and/or shape after deformation by stress or a biasing force. The thermoplastic-based elastomer may be or include any one or more suitable thermoplastic elastomers. For example, the thermoplastic-based elastomer may be or include a single thermoplastic elastomer. In another example, the thermoplastic-based elastomer may be or include a plurality (e.g., two or more) of thermoplastic elastomers. The plurality of thermoplastic elastomers may be a homogenous or heterogeneous mixture or blend. The thermoplastic elastomer may have crosslinks capable of or configured to prevent permanent deformation during use via increased elasticity and/or decreased plasticity. The one or more thermoplastic elastomers may have one or more crosslinks about 4,000 to about 10,000 monomer units in the respective polymeric chain.

In an exemplary implementation, the thermoplastic interlayer 106 may include one or more thermoplastic polyurethanes and/or thermoplastic polyurethane elastomers. For example, the thermoplastic elastomer may be or include a blend of two or more thermoplastic polyurethanes and/or thermoplastic polyurethane elastomers. The thermoplastic polyurethane and/or thermoplastic polyurethane elastomer may include a block copolymer including a hard segment and a soft segment. The hard segment may include a polyisocyanate (e.g., diisocyanate), a chain extender, or a combination thereof. The chain extender may be or include, are not limited to, one or more of a polyol (e.g., a diol, a glycol, etc.), a polyfunctional amine (e.g., a primary amine, a secondary amine, or the like, or a combination thereof), or the like, or a combination thereof. For example, the chain extender may be or include, but is not limited to, one or more of a 1,4-butanediol, a glycol, a diol, an ethylene glycol, a diamine, or the like, or a combination thereof. The soft segment may include a long chain diol, a long chain elastomeric polyol, or a combination thereof. Illustrative long chain diols and long chain elastomeric polyols may be or include, but are not limited to, one or more of a polyether, a polyether polyol, a polcaprolactone polyester, a polyadipate polyester, a polytetramethylene glycol ether, a hydroxyl group end-capped monomer, a hydroxyl group end-capped, a polyester, a polyester polyol, or the like, or a combination thereof. Illustrative polyether polyols may be or include, but are not limited to, a diol and/or a triol having a molecular weight of from about 4,000 Da to about 6,000 Da or more. Illustrative polyesters may be or include, but are not limited to a polyester prepared from a glycol, such as an ethylene glycol, and an adipic acid having a molecular weight of from about 1,000 Da to about 3,000 Da and/or a poly(epsilon-caprolactone). Illustrative polyesters may also be or include, but are not limited to a polyester prepared from a polycarboxylic acid, such as a dicarboxylic acid (e.g., an adipic acid) and a polyol. The polyester may have a hydroxy moiety at one or more termini. In an exemplary implementation, the thermoplastic polyurethane and/or thermoplastic polyurethane elastomer may be a reaction product or prepared from reacting one or more diisocyanates with one or more long chain diols and/or the chain extender. The thermoplastic polyurethane and/or thermoplastic polyurethane elastomer may include one or more crosslinks.

It should be appreciated that the type of the soft segment polyol utilized to prepare the thermoplastic polyurethane and/or thermoplastic polyurethane elastomer may at least partially determine or facilitate the classification thereof. For example, the soft segment polyol of the thermoplastic polyurethane and/or thermoplastic polyurethane elastomer may classify the thermoplastic as a polyether-based thermoplastic, a polyester-based thermoplastic, a polycaprolactam-based thermoplastic, or the like. Particularly, a polyether polyol and a polyester polyol may be utilized as the polyol of the soft segment to prepare a polyether-based thermoplastic and a polyester-based thermoplastic, respectively. A polyester-based thermoplastic polyurethane may include, but is not limited to, a hydroxy terminated polyester polyol having a molecular weight of up to about 2,500 Da, about 3,000 Da, about 3,500 Da, about 4,000 Da, or more. The polyester-based thermoplastic polyurethane may be prepared by step growth and/or condensation polymerization, as is conventionally known in the art. The polyether polyol of a polyether-based thermoplastic polyurethane may be prepared using an epoxide by addition, ring opening, and/or anionic polymerization. The “hard segment” of the thermoplastic polyurethane elastomer may include a “chain extender” polyol, which may include a short chain diol, such as, a 1,4-butanediol, a 1,6-hexanediol, and/or an ethylene glycol, and a diisocyanate, such as, a 4,4′-diphenylmethane diisocyanate (MDI), a hydrogenated 4,4′-diphenylmethane diisocyanate (HMDI), a hexamethylene diisocyanate (HDI), a toluene diisocyanate (TDI), and/or a 1,5-diisocyanate, or the like, or any combination thereof. The hard segment may be capable of or configured to hydrogen bond with a different chain to promote or facilitate crystallization. Illustrative thermoplastic polyurethanes may be or include, but are not limited to, one or more of an aromatic polycaprolactam polyurethane, an aliphatic polycaprolactam polyurethane, an aromatic polyester-based thermoplastic polyurethane, a linear polyester-based thermoplastic polyurethane, an aliphatic polyester-based polyurethane, a linear polyether-based polyurethane, an aliphatic polyether-based polyurethane, an aromatic polyether-based polyurethane, or a combination thereof.

In at least one implementation, the thermoplastic interlayer 106 may include one or more of a polyether-based thermoplastic polyurethane, an aliphatic polyether thermoplastic polyurethane, a thermoplastic polyurethane resin blend, or the like, or any combination thereof. In an exemplary implementation, the thermoplastic interlayer 106 may include a polyether-based thermoplastic polyurethane. Illustrative polyether-based thermoplastic polyurethanes may be or include, but are not limited to, one or more of ESTANE AG-8451 TPU (an aliphatic polyether TPU commercially available from Lubrizol Corporation of Wickliffe, OH), TEXIN® 8980D (an aliphatic polyether TPU commercially available from Covestro, LLC), TECOFLEX® EG-72D (an aliphatic polyether TPU commercially available from Lubrizol Corporation of Wickliffe, OH), ELASTOLLAN® (a class of thermoplastic polyurethanes commercially available from BASF of Ludwigshafen, Germany), or the like, or any combination thereof. In an exemplary implementation, the thermoplastic interlayer 106 includes an ELASTOLLAN® TPU, such as a polyester based TPU, a polyether based TPU, or a combination thereof. The ELASTOLLAN® TPUs may include aliphatic thermoplastic polyurethanes, polyester-based TPU, polyether-based TPU, glass fiber-reinforced TPU, TPU based on renewable resources, or any combination thereof. The ELASTOLLAN® TPUs may have a Shore A hardness of from about 35 to about 98. The ELASTOLLAN® TPUs may also have a Shore D hardness of from about 47 to about 83. The ELASTOLLAN® TPUs may be free or substantially free of lubricants. In an exemplary implementation, the thermoplastic interlayer 106 includes ELASTOLLAN® L 1275 A 10, which is an aliphatic polyether-based thermoplastic polyurethane having a Shore A hardness of about 75 and being free of lubricants and additives.

In an exemplary implementation, the thermoplastic interlayer 106 includes ESTANE® AG-8451. The polyether-based thermoplastic polyurethane may have a specific gravity, as determined according to reference test ASTM D792 of the American Society for Testing and Materials (ASTM), of from about 1 g/cc to about 1.2 g/cc, or about 1.08 g/cc. The polyether-based thermoplastic polyurethane may have a tensile strength, as determined according to reference test ASTM D412, of from about 40 MPa to about 60 MPa, or about 48 MPa. The polyether-based thermoplastic polyurethane may have an elongation at break, as determined according to reference test ASTM D412, of from about 450% to about 500%, or about 475%. The polyether-based thermoplastic polyurethane may have a 100% modulus, as determined according to reference test ASTM D412, of from about 3 MPa to about 4 MPa, or about 3.45 MPa. The polyether-based thermoplastic polyurethane may have a tear strength, as determined according to reference test ASTM D624, of from about 50 kN/m to about 60 kN/m, or about 56 kN/m. The polyether-based thermoplastic polyurethane may have a glass transition temperature (Tg), as determined with a differential scanning calorimeter (DSC), of from about −65 °C to about −55 °C, or about 60 °C. The polyether-based thermoplastic polyurethane may have a refractive index, as determined according to reference test ASTM D542-95, of from about 1 to about 2, about 1.25 to about 1.75, or about 1.5. The polyether-based thermoplastic polyurethane may have a haze, as determined according to reference test ASTM D1003, of from about 0% to about 1%, about 1% to about 0.5%, or about 0.3%. The polyether-based thermoplastic polyurethane may have a yellow index, as determined according to reference test ASTM D1925, of less than or equal to about 1.5%, or less than or equal to about 1%. The polyether-based thermoplastic polyurethane may have a visible transmission, as determined according to reference test ASTM D1003, of from about 80% to about 100% or about 85% to about 95%. The polyether-based thermoplastic polyurethane may have a thermomechanical analysis (TMA) peak of from about 85 °C to about 90 °C, or about 88 °C. The polyether-based thermoplastic polyurethane may have a thermomechanical analysis (TMA) range of from about 65 °C to about 130 °C.

In at least one implementation, the thermoplastic interlayer 106 includes one or more of a polyester-based thermoplastic polyurethane and/or an elastomer thereof. The polyester-based thermoplastic polyurethane and/or elastomer may be or include crystalline and/or semi-crystalline polymers. The plurality of polyester-based thermoplastic polyurethane and/or elastomer may include one or more of an aromatic polyester-based thermoplastic polyurethane, a linear polyester-based thermoplastic polyurethane, an aliphatic polyester-based polyurethane, an elastomer thereof, or a combination thereof. Illustrative polyester-based thermoplastic polyurethane and/or elastomer may be or include, but are not limited to, those commercially available from Lubrizol Corporation of Wickliffe, Ohio under the general designation ESTANE® and/or PEARLSTICK™. For example, the polyester-based thermoplastic polyurethane may be or include one or more of the following: ESTANE® 5701, which is a polyester-based polyurethane polymer; ESTANE® 5701 F1, which is an aromatic polyester-based polyurethane polymer; ESTANE® 5702, which is an aromatic polyester-based polyurethane polymer; ESTANE® 5703, which is an aromatic polyester based polyurethane polymer; ESTANE® 5707 F1, which is an aromatic polyester-based polyurethane polymer; ESTANE® 5712 F30, which is an aromatic polyester-based polyurethane polymer; ESTANE® 5713 F2, which is an aromatic polyester-based polyurethane; ESTANE® 58213 NAT, which is a polyester based thermoplastic polyurethane; ESTANE® 5715, which is an aromatic polyester-based thermoplastic polyurethane; PEARLSTICK® 5715 F2, which is an aromatic polyester-based thermoplastic polyurethane; PEARLSTICK® 5715, which is an aromatic polyester-based thermoplastic polyurethane, PEARLSTICK® 5701, which is a polyester-based thermoplastic polyurethane, or the like, or any combination thereof.

The thermoplastic interlayer 106 may have a thickness suitable for or sufficient to adhere the first and second outer layers 102, 104 with one another. For example, the thermoplastic interlayer 106 may have a thickness of at least about 254 μm (about 10 Mils) to about 1524 μm (about 60 Mils) or more. In an exemplary implementation, the thermoplastic interlayer 106 may have a thickness of from about 254 μm (about 10 Mils) to about 1524 μm (about 60 Mils) or more, about 254 μm (about 10 Mils) to about 1015 μm (40 Mils), about 254 μm (about 10 Mils) to about 762 μm (about 30 Mils), about 254 μm (about 10 Mils) to about 508 μm (about 20 Mils), or about 381 μm (about 15 Mils). In another example, the thermoplastic interlayer 106 may have a thickness of from about 254 μm, about 381 μm, or about 508 μm to about 762 μm, about 1015 μm, about 1270 μm, or about 1524 μm. In an exemplary implementation, the thermoplastic interlayer 106 has a thickness of from about 254 μm (about 10 Mils) to about 508 μm (about 20 Mils) or about 381 μm (about 15 Mils). It should be appreciated that the thickness may be determined according to conventional methods. For example, the thickness may be determined with a micrometer.

In other embodiments, the thermoplastic interlayer 106 may be used for skin-contact applications, such as elastic or adhesive bandages, dressings, wound dressings, compression bandages, surgical dressings, Tulle dressings, alginate dressings, foam dressings, hydrogel dressings, hydrocolloid dressings, gauze rolls, gauze pads and the like. In these embodiments, the thermoplastic layer 106 may have a thickness of at least about 254 μm (about 1 Mil) to about 1524 μm (about 10 Mils) or more. In an exemplary implementation, the thermoplastic layer 106 may have a thickness of from about 25.4 μm (about 1 Mil) to about 254 μm (about 10 Mils) or more, about 38.2 μm (about 1.5 Mils) to about 127 μm (5 Mils), about 46 μm (about 1.8 Mils) to about 76.2 μm (about 3 Mils), or about 50.8 μm (about 2 Mils).

The thermoplastic interlayer 106 may include an adhesion promoter capable of or configured to enhance the coupling, bonding, or adhesion between the outer layers 102, 104 and the thermoplastic interlayer 106, and prevent delamination from the outer layers 102, 104. Illustrative adhesion promoters may be or include, but are not limited to, organosilanes, organotitanates, zirconates, zircoaluminates, alkyl phosphate esters, metal organics, or the like, or a combination thereof.

The thermoplastic interlayer 106 may have an index of refraction that may be within about 30% of the index of refraction of the outer layers 102, 104. For example, the thermoplastic interlayer 106 may have an index of refraction that is within about 30% or less, about 25% or less, about 20% or less, about 15% or less, or about 10% or less of the index of refraction of the outer layers 102, 104 (e.g., glass).

The laminate 100 and/or the components thereof may be at least partially transparent, semi-transparent, substantially transparent, or transparent. For example, the laminate 100, the thermoplastic interlayer 106, the first outer layer 102, the second outer layer 104, or any combination thereof may be transparent, at least partially transparent or at least semi-transparent. For example, the laminate 100, the thermoplastic interlayer 106, the first outer layer 102, the second outer layer 104, or any combination thereof may have a light transmission or visible light transmission (VLT), as measured according to reference test ASTM-D1003 of the American Society for Testing and Materials (ASTM), of greater than or equal to about 30%, greater than or equal to about 50%, greater than or equal to about 70%, greater than or equal to about 75%, greater than or equal to 80%, greater than or equal to 82%, greater than or equal to 84%, greater than or equal to 86%, greater than or equal to 88%, greater than or equal to 90%, greater than or equal to 95%, greater than or equal to 98%, or greater than or equal to 99%. The laminate 100, the thermoplastic interlayer 106, the first outer layer 102, the second outer layer 104, or any combination thereof may at least partially filter visible light to thereby avoid imparting unnatural color to the transmitted light.

The laminate 100, the thermoplastic interlayer 106, the first outer layer 102, the second outer layer 104, or any combination thereof may have an ultraviolet to visible spectrum transmission (UV-VIS), as measured with a UV-VIS spectrophotometer, of less than or equal to about 10%, less than or equal to about 5%, or less than or equal to about 1%.

The laminate 100, the thermoplastic interlayer 106, the first outer layer 102, the second outer layer 104, or any combination thereof may have an ultraviolet transmission, as measured with a UV-VIS spectrophotometer, of less than or equal to about 10%, less than or equal to about 5%, or less than or equal to about 1%.

The laminate 100, the thermoplastic interlayer 106, the first outer layer 102, the second outer layer 104, or any combination thereof may have a haze, as measured according to reference test ASTM-D1003, of greater than or equal to 0% and less than or equal to about 10%. For example, any one of the laminate 100, the thermoplastic interlayer 106, the first outer layer 102, the second outer layer 104, or any combination thereof may have haze, as measured according to ASTM-D1003 of from 0% to less than or equal to about 10%, less than or equal to about 8%, less than or equal to about 6%, less than or equal to about 5%, less than or equal to about 4%, less than or equal to about 3%, less than or equal to about 2%, or less than or equal to about 1%.

The thermoplastic interlayer 106 may include one or more functional elements contained, dispersed, mixed, or otherwise disposed therein. Illustrative functional elements may be or include, but are not limited to, one or more of solar control elements, luminophores, ionomers, optical elements, ultraviolet (UV) agents UV absorbers, UV reflectors, infrared (IR) absorbers, IR reflectors, light stabilizers, or the like, or any combination thereof. In an exemplary implementation, the thermoplastic interlayer 106 includes a combination of one or more UV blockers, one or more light stabilizers, or a combination thereof disposed therein.

The one or more functional elements may be present in the thermoplastic interlayer 106 in an amount of from greater than or equal to 0 wt % to about 10 wt %, or about 0.01 wt % to about 9 wt %, or about 0.05 wt % to about 8 wt %, or about 0.2 wt % to about 7 wt % of about 0.5 wt % to about 5 wt %, or about 1 wt % to about 4 wt %, based on the total weight of the thermoplastic interlayer 106. For example, any one or more of the functional elements may, each or combined, be present in an amount of from greater than or equal to 0 wt %, greater than or equal to about 0.01 wt %, greater than or equal to about 0.05 wt %, greater than or equal to about 0.1 wt %, greater than or equal to about 0.15 wt %, greater than or equal to about 0.2 wt %, greater than or equal to about 0.5 wt %, greater than or equal to about 1 wt %, greater than or equal to about 2 wt %, greater than or equal to about 3 wt %, or greater than or equal to about 4 wt % to less than or equal to about 5 wt %, less than or equal to about 6 wt %, less than or equal to about 7 wt %, less than or equal to about 8 wt %, less than or equal to about 9 wt %, or less than or equal to about 10 wt %, based on the total weight of the thermoplastic interlayer 106.

The thermoplastic interlayer 106 and/or the components thereof may be capable of or configured to allow, reflect, absorb, and/or block the transmission of electromagnetic radiation directed therethrough. For example, the thermoplastic interlayer 106, the one or more thermoplastic polymers thereof, and/or the one or more functional elements may be capable of or configured to allow, reflect, absorb, and/or block the transmission of electromagnetic radiation directed therethrough.

In at least one implementation, the thermoplastic interlayer 106, the one or more thermoplastic polymers thereof, and/or the one or more functional elements may be capable of or configured to allow the transmission of electromagnetic radiation having a wavelength of from greater than or equal to about 400 nm. In another implementation, the thermoplastic interlayer 106, the one or more thermoplastic polymers thereof, and/or the one or more functional elements may be capable of or configured to reflect and/or block the transmission of electromagnetic radiation having a wavelength of less than or equal to about 400 nm. In an exemplary implementation, the thermoplastic interlayer 106, the one or more thermoplastic polymers thereof, and/or the one or more functional elements may be capable of or configured to block at least 90%, at least 92%, at least 94%, or at least 95% of electromagnetic radiation having a wavelength of from about 100 nm to about 400 nm, from about 100 nm to about 410 nm, from about 380 nm to about 400 nm, or from about 380 nm to about 410 nm. In at least one implementation, the thermoplastic interlayer 106, the one or more thermoplastic polymers thereof, and/or the one or more functional elements may be capable of or configured to block greater than or equal to about 95%, greater than or equal to about 98%, greater than or equal to about 99%, about 99.5%, or about 99.9% of electromagnetic radiation having a wavelength of from about 380 nm to about 400 nm. In at least one implementation, the thermoplastic interlayer 106, the one or more thermoplastic polymers thereof, and/or the one or more functional elements may be capable of or configured to block greater than or equal to about 98%, greater than or equal to about 99%, greater than or equal to about 99.5%, greater than or equal to about 99.9% of electromagnetic radiation (e.g., light) having a wavelength of about 400 nm. In an exemplary implementation, the thermoplastic interlayer 106, the one or more thermoplastic polymers thereof, and/or the one or more functional elements may be capable of or configured to block at least 99% of the electromagnetic radiation having a wavelength of from about 380 nm to about 400 nm.

The thermoplastic interlayer 106 and/or one or more components thereof may have a yellowness index, as determined according to reference test ASTM D1925 of the American Society for Testing and Materials (ASTM), of less than or equal to about 3%, less than or equal to about 2.5%, less than or equal to about 2%, less than or equal to about 1.5%, or less than or equal to about 1%.

The thermoplastic interlayer 106 may have a thickness of from greater than or equal to about 5 Mils to less than or equal to about 40 Mils. For example, the thermoplastic interlayer 106 may have a thickness of from about 5 Mils, about 10 Mils, about 15 Mils, or about 20 Mils to about 25 Mils, about 30 Mils, about 35 Mils, or about 40 Mils. In another example, the thermoplastic interlayer 106 may have a thickness of from about 5 Mils to about 40 Mils, about 10 Mils to about 35 Mils, about 15 Mils to about 30 Mils, or about 20 Mils.

In an exemplary implementation, the one or more functional elements may be or include, but are not limited to, one or more UV agents, such as one or more UV absorbers. The one or more UV absorbers may be or include, but are not limited to, one or more of the benzotriazole family, one or more of the triazine family, one or more of the benzophenone family, one or more of the benzylidene malonate family, one or more of the imidazole family, one or more of the cyanoacetate family, or any combination thereof. In an exemplary implementation, the one or more UV absorbers may exclude, be free, or substantially free of materials and substances identified by the Registration, Evaluation, Authorization, and Restriction of Chemicals (REACH), including those materials identified or listed as a Substance of Very High Concern (SVHC), a Restricted Substance (Annex XVII of REACH), a Substance Subject to Authorization (Annex XIV of REACH), a Substance Subject to Registration, a Persistent Organic Pollutant (POPs), Nanomaterials, a Candidate List Substance (Pre-SVHC stage), or any combination thereof.

The UV absorbers of the benzotriazole family may be represented by formula (I):

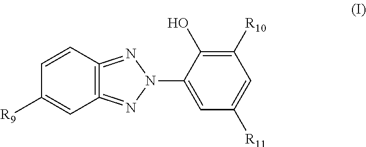

where each of R₉, R₁₀, and R₁₁ may individually or independently be a hydrogen, a halogen, or a group represented by CₐH_{b}N_{c}O_{d}Sₑ, where each of a, b, c, d, and e may independently be from 0 to 30. Illustrative UV absorbers of the benzotriazole family may be or include, but are not limited to, one or more of 2-(2H-benzotriazol-2-yl)-4,6-bis(1,1-dimethylpropyl)-phenol; phenol, 2,2′-methylene-bis(6-(2H-benzotriazol-2-yl)-4-(1,1,3,3-tetramethyl-butyl)); 2-(2′-Hydroxy-3′,5′-di-t-amylphenyl)benzotriazole; 2-Hydroxy-4-methoxybenzophenone; 2-[2-hydroxy-3,5-di(1,1-dimethylbenzyl)phenyl]; 2-(5-tert-Butyl-2-hydroxyphenyl)-2H benzotriazole; 2-(2-hydroxy-5-methylphenyl)benzotriazole; 2-(2H-Benzotriazol-2-yl)-4-(1,1,3,3-tetramethylbutyl) phenol; 2,4-Di-tert-butyl-6-(5-chloro-2H-benzotriazol-2-yl) phenol; 2-(2′-Hydroxy-3′,5′-di-tert-butylphenyl)benzotriazole; 3-(2H-Benzotriazolyl)-5-(1,1-di-methylethyl)-4-hydroxy-benzenepropanoic acid octyl esters; methyl 3-[3-tert-butyl-4-hydroxy-5-(5-chloro-2H-benzotriazol-2-yl)phenyl]propionate; 2-(2H-Benzotriazol-2-yl)-6-(1-methyl-1-phenylethyl)-4-(1,1,3,3-tetramethylbutyl) phenol; reaction products of methyl 3-(3-(2H-benzotriazole-2-yl)-5-t-butyl-4-hydroxyphenyl) propionate/PEG 300; 2-(2′-Hydroxy-5′-(2-hydroxyethyl))-benzotriazole; 2-(2′-Hydroxy-5′methacryloxyethylphenyl)-2H-benzotriazole; 2-[4,6-Bis(2,4-dimethylphenyl)-1,3,5-triazin-2-yl]-5-(octyloxy) phenol; or any combinations thereof.

Illustrative UV absorbers of the benzophenone family may be or include, but are not limited to, one or more of 2,4-dihydroxy benzophenone; 2-hydroxy-4-methoxybenzophenone; 2-hydroxy-4-n-(octyloxy)benzophenone; 2,2′,4,4′-tetrahydroxybenzophenone; 2,2′-dihydroxy-,4,4′-dimethoxy benzophenone; sulisobenzone; 2-hydroxy-4-n-octoxybenzophenone; 2,2′-dihydroxy-4-methoxy benzophenone; 2-hydroxy-4-methoxybenzophenone; 2,2′-dihydroxy-4,4′-dimethoxy benzophenone; 2,2′,4,4′-tetrahydroxybenzophenone; or any combination thereof.

Illustrative UV absorbers of the triazine family may be or include, but are not limited to, one or more of 2-(4,6-diphenyl-1,3,5-triazin-2-yl)-5-[(hexyl)oxy]-phenol; 2,4-diphenyl-6-(2-hydroxy-4-methoxyphenyl)-1,3,5-triazine; 2,4-diphenyl-6-(2-hydroxy-4-ethoxyphenyl)-1,3,5-triazine; 2,4-diphenyl-(2-hydroxy-4-propoxyphenyl)-1,3,5-triazine; 2,4-diphenyl-(2-hydroxy-4-butoxyphenyl)-1,3,5-triazine; 2,4-diphenyl-6-(2-hydroxy-4-butoxyphenyl)-1,3,5-triazine; 2,4-diphenyl-6-(2-hydroxy-4-hexyloxyphenyl)-1,3,5-triazine; 2,4-diphenyl-6-(2-hydroxy-4-octyloxyphenyl)-1,3,5-triazine; 2,4-diphenyl-6-(2-hydroxy-4-dodecyloxyphenyl)-1,3,5-Triazine; 2,4-diphenyl-6-(2-hydroxy-4-benzyloxyphenyl)-1,3,5-triazine; or any combination thereof.

Illustrative UV absorbers of the benzylidene malonate family may be or include, but are not limited to, one or more of propanedioic acid [(4-methoxyphenyl)-methylene]-dimethyl ester).

Illustrative UV absorbers of the imidazole family may be or include, but are not limited to, one or more of (2,3-Dimethylphenyl) (1H-imidazol-4-yl) methanone; (2,3-Dimethylphenyl) (1H-imidazol-5-yl) methanone; 4-(2′,3′-Dimethylbenzoyl) imidazole; (2,3-Dimethylphenyl) (1-(triphenylmethyl)-1H-imidazol-4-yl) methanone; (2,3-dimethylphenyl) (1-trityl-1h-imidazol-4-yl) methanone; (2,3-Dimethylphenyl) [1-(triphenylmethyl)-1H-imidazol-4-yl]methanone, derivatives thereof, salts thereof, compounds thereof, or the like, or any combination thereof. In an exemplary implementation, the UV absorber includes one or more of (2,3-Dimethylphenyl) (1H-imidazol-4-yl) methanone [CAS No. 91874-85-0], (2,3-Dimethylphenyl) (1-(triphenylmethyl)-1H-imidazol-4-yl) methanone [CAS No. 176721-02-1], or any combination thereof.

The UV absorbers of the cyanoacetate family may be represented by formula (II):

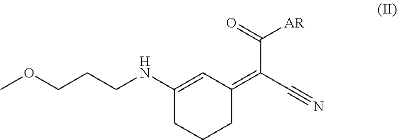

where A may be —O— or —NH—, R may be a C1-C22 alkyl group, a C2-C22 alkenyl group, a C2-C22 alkynyl group, a C3-C22 cycloalkyl group, or a C3-C22 cycloalkenyl group, where each of the groups may be interrupted by one or more oxygen (—O—). It should be appreciated that all isomers are contemplated. For example, the E/E or E/G geometric isomers of formula (II) are contemplated. Illustrative UV absorbers of the cyanoacetate family may be or include, but are not limited to, one or more of a merocyanine; methoxypropylamino cyclohexenylidene ethoxyethylcyanoacetate (MCE); 2-ethoxyethyl(2Z)-2-cyano-2-[3-(3-methoxypropylamino)cyclohex-2-en-1-ylidene]acetate; 2-ethoxyethyl 2-cyano-2-[(1Z)-3-[(3-methoxypropyl)amino]cyclohex-2-en-1-ylidene]acetate; ethyl(2Z)-cyano {3-methoxypropyl)amino]cyclohex-2-en-1-ylidene}ethanoate; (2Z)-2-cyano-N-(3-methoxypropyl)-2-{3-[(3-methoxypropyl)amino]cyclohex-2-en-1-ylidene}ethanamide; 2-ethoxyethyl(2Z)-cyano {3-[(3-methoxypropyl)amino]cyclohex-2-en-1-ylidene}ethanoate; 2-methylpropyl(2Z)-cyano {3-[(3-methoxypropyl)amino]cyclohex-2-en-1-ylidene}ethanoate; 2-butoxyethyl(2Z)-cyano {3-[(3-methoxypropyl)amino]cyclohex-2-en-1-ylidene}ethanoate; 3-methoxypropyl(2Z)-cyano {3-[(3-methoxypropyl)amino]cyclohex-2-en-1-ylidene}ethanoate; 3-ethoxypropyl(2Z)-cyano {3-[(3-methoxypropyl)amino]cyclohex-2-en-1-ylidene}ethanoate, or the like, or isomers thereof, derivatives thereof, or any combination thereof. In an exemplary implementation, the UV absorber includes methoxypropylamino cyclohexenylidene ethoxyethylcyanoacetate (MCE) [CAS No. 1419401-88-9].

In at least one implementation, the thermoplastic interlayer 106 includes a single UV absorber. In another implementation, the thermoplastic interlayer 106 includes a combination or plurality of UV absorbers. For example, the thermoplastic interlayer 106 may include any combination of the UV absorbers described herein.

In another embodiment, the thermoplastic interlayer 106 may include a first UV absorber of the imidazole family and a second UV absorber of the cyanoacetate family. In another example, the thermoplastic interlayer 106 may include a first UV absorber of the imidazole family and a second UV absorber of the imidazole family. In yet another example, the thermoplastic interlayer 106 may include a first UV absorber of the cyanoacetate family and a second UV absorber of the cyanoacetate family. It should be appreciated that the combination or plurality of UV absorbers may include a first UV absorber, second UV absorber, third UV absorber, fourth UV absorber, fifth UV absorber, sixth UV absorber, or more.

In another embodiment, the thermoplastic interlayer 106 may include a combination of at least one UV absorber of the imidazole family and/or the cyanoacetate family, and another or second UV absorber of the benzotriazole family, the triazine family, the benzophenone family, the benzylidene malonate, or any combination thereof. In]another example, the thermoplastic interlayer 106 may include a combination of at least one UV absorber of the imidazole family and another UV absorber of the benzotriazole family, the triazine family, the benzophenone family, the benzylidene malonate, the cyanoacetate family, or any combination thereof. In another example, the thermoplastic interlayer 106 may include a combination of at least one UV absorber of the cyanoacetate family and another UV absorber of the benzotriazole family, the triazine family, the benzophenone family, the benzylidene malonate, the imidazole family, or any combination thereof.

The one or more UV absorbers may be present in the thermoplastic interlayer 106 in an amount of about 0.1 wt % to about 5.0 wt %, or about 0.1 wt % to about 2.0 wt %, or about 0.2 wt % to about 1.5 wt %, or about 0.25 wt % to about 1.0 wt % of a total weight percentage of the thermoplastic polyurethane layer. The thermoplastic polyurethane may include about 99.9 wt % to about 95 wt %, or about 99.9 wt % to about 98 wt %, or about 99.8 wt % to about 98.5 wt %, or about 99.75 wt % to about 99 wt % a total weight percentage of the thermoplastic polyurethane layer.

In embodiments, the UV blocker includes a material in the imidazole family in about 0.1 wt % to about 0.5 wt %, or about 0.2 wt % to about 0.3 wt % or about 0.25 wt % of a total weight percentage of the thermoplastic polyurethane layer.

In embodiments, the UV blocker includes a material in the cyanoacetate family in about 0.5 wt % to about 5.0 wt %, or about 0.75 wt % to about 2.0 wt % or about 1.0 wt % of a total weight percentage of the thermoplastic polyurethane layer.

The weight or volume ratio of one (first) UV absorber to another (second) UV absorber may be from about 10:1 (i.e., about 10 to about 1) to about 1:10. For example, the weight or volume ratio of a first UV absorber to a second UV absorber may be from about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, or about 1:10.

In at least one implementation, at least one of the UV absorbers of the thermoplastic interlayer 106 may be a concentrate in a base resin or a base polymer. For example, at least one of the UV absorbers (e.g., first, second, third, etc.) may be a concentrate that may be combined, mixed, dispersed, disposed, or otherwise contacted with the base resin or the base polymer. The base resin or base polymer may be or include any one or more of the thermoplastic polymers described herein. The weight ratio of the UV absorber concentrate to the base resin or base polymer may be from about 20:1 to about 3:1. The loading of the concentrate in the base resin may be from about 0.1 wt % to about 10 wt %, based on the total weight thereof.

In at least one implementation, the thermoplastic interlayer 106 may include one or more light stabilizers. The light stabilizer may be capable of or configured to protect the polymers of the laminate 100 and/or the thermoplastic interlayer 106 thereof from photooxidation caused by exposure to UV radiation, provide thermal stability to the laminate 100 and/or the thermoplastic interlayer 106 thereof, or any combination thereof. Illustrative light stabilizers may be or include, but are not limited to, one or more of a hindered amine light stabilizer (HALS), a light stabilizer prepared by combining bis(1,2,2,6,6-pentamethyl-4-piperidyl) sebacate with methyl 1,2,2,6,6-pentamethyl-4-piperidyl sebacate; bis-(2,2,6,6-tetramethyl-4-piperidinyl) sebacate; bis-(1,2,2,6,6-pentamethyl-4-piperidinyl)-2-n-butyl-2-(3,5-di-tert-butyl-4-hydroxybenzyl) malonate; propanedioic acid, [(4-methoxyphenyl)-methylene]-bis-(1,2,2,6,6-pentamethyl-4-piperidinyl) ester; about 10 wt % of dimethyl succinate polymer with 4-hydroxy-2,2,6,6,-tetramethyl-1-piperidineethanol and 90 wt % of N,N′″-[1,2-ethanediylbis [[4,6-bis [butyl(1,2,2,6,6-pentamethyl-4-piperidinyl)amino]-1,3,5-traizin-2-yl]imino]-3,1-propanediyl]]bis [N′N″-dibutyl-N′N″-bis(1,2,2,6,6-pentamethyl-4-piperidinyl)]-I; or combinations thereof. In an exemplary implementation, the thermoplastic interlayer 106 includes at least one light stabilizer, where the at least one light stabilizer may be the combination or reaction product of bis(1,2,2,6,6-pentamethyl-4-piperidyl) sebacate combined with methyl 1,2,2,6,6-pentamethyl-4-piperidyl sebacate, which is commercially available as CHISORB® 292 from Double Bond Chemicals Ind., Co., Ltd. of Shanghai, China, EVERSORB® 93 from Everlight Chemical of Taipei, Taiwan, RIASORB® UV-292 from Rianion Corp., or any combination thereof.

The one or more stabilizers may be present in the thermoplastic interlayer 106 in an amount of from about 0.01 wt % to about 2 wt %, based on the total weight of the thermoplastic interlayer 106. For example, the one or more stabilizers may be present in an amount of from about 0.01 wt %, about 0.02 wt %, about 0.03 wt %, about 0.04 wt %, about 0.05 wt %, about 0.8 wt % or about 1 wt % to about 1.2 wt %, about 1.4 wt %, about 1.6 wt %, about 1.8 wt %, or about 2 wt %.

FIG. 2 illustrates a schematic cross-sectional view of a laminate 200, according to one or more implementations. The laminate 200 may be similar in some respects to the laminate 100 described above and therefore may be best understood with reference to the description of FIG. 1 where like numerals designate like components and will not be described again in detail. The laminate 200 may include the first outer layer 102, the second outer layer 104, and the thermoplastic interlayer 106 interposed between the first and second outer layers 102, 104. As illustrated in FIG. 2, the thermoplastic interlayer 106 may be disposed directly adjacent the first outer layer 102. It should be appreciated that the thermoplastic interlayer 106 may be disposed anywhere throughout the laminate 200. For example, the thermoplastic interlayer 106 may be disposed adjacent the second outer layer 104 or adjacent any other layer further described herein.

In at least one implementation, the laminate 200 may include an infrared (IR cutoff layer) 202 interposed between the first and second outer layers 102, 104. As illustrated in FIG. 2, the IR cutoff layer 202 may be disposed adjacent the thermoplastic interlayer 106. The IR cutoff layer 202 may be a transparent coating capable of or configured to absorb or block infrared electromagnetic radiation. The IR cutoff layer 202 may be a coating formed by conventional deposition, such as sputter deposition, plasma deposition, or the like. The IR cutoff layer 202 may include a transparent electromagnetic interference (EMI) shielding disposed therein and/or thereon. The IR cutoff layer 202 may include a transparent antenna disposed therein and/or thereon. The transparent antenna may be formed from semiconductor nanowires or a metal (e.g., silver) nanowires. The transparent antenna may be capable of or configured to protect the technical features built into a vehicle from crosstalk and/or other interference. While the IR cutoff layer 202 may include the EMI shielding and/or the transparent antenna, it should be appreciated that each of the IR cutoff layer 202, the EMI shielding, and/or the transparent antenna may be disposed or formed as two or more separate layers.

In at least one implementation, the laminate 200 may include a PET layer or film 204. The PET layer 204 may be interposed between the first and second outer layers 102, 104. For example, as illustrated in FIG. 2, the PET layer 204 may be disposed between the outer layers 102, 104 and adjacent the IR cutoff layer 202. The PET layer 204 may be capable of or configured to provide a substrate for deposition of the IR cutoff layer 202, the transparent antenna, the EMI shielding, or any combination thereof. For example, the cutoff layer 202, the transparent antenna, and/or the EMI shielding may be disposed on the PET layer 204. The PET layer 204 may also provide additional structural support or integrity to the laminate 200. The PET layer 204 may have a thickness of from about 15 μm to about 40 μm, about 20 μm to about 30 μm, or about 25 μm.

In at least one implementation, the laminate 200 may include additional thermoplastic interlayers or thermoplastic layers 206, 210. The thermoplastic interlayers 206, 210 may be similar to the thermoplastic interlayer 106 of FIG. 1. In at least one implementation, each of the thermoplastic interlayers 206, 210 may independently include or exclude any one or more of the functional elements. For example, the thermoplastic interlayer 206 may include one or more UV absorbers and one or more light stabilizers. In another example, the thermoplastic interlayer 210 may be free or substantially free of UV absorbers and/or light absorbers. The thermoplastic layer or interlayers 106, 206, 210 may be capable of or configured to function as a substrate for any one or more of the other layers of the laminate 100.

In at least one implementation, the laminate 200 may include a low emissivity (low-E) layer 208 interposed between the first and second outer layers 102, 104. As illustrated in FIG. 2, the low-E layer 208 may be interposed between the additional thermoplastic interlayers 206, 210. The low-E layer 208 may be formed by conventional deposition. For example, the low-E layer 212 may be sputter coated on the thermoplastic interlayers 206, 210.

While FIG. 2 illustrates the laminate 200 including the thermoplastic interlayer 106, the IR cutoff layer 202, the PET layer 204, the TPU layers 206, 210, and the low-E layer 208, it should be appreciated that the laminate 200 may include one or more of the thermoplastic interlayer 106, the IR cutoff layer 202, the PET layer 204, the TPU layers 206, 210, the low-E layer 208, or any combination thereof. For example, the laminate 200 may include more than one of the low-E layers 208. In another example, the laminate 200 may exclude any one or more of the foregoing components. It should further be appreciated that while FIG. 2 illustrates a particular combination and/or order of the thermoplastic interlayer 106, the IR cutoff layer 202, the PET layer 204, the TPU layers 206, 210, and the low-E layer 208, the laminate 200 is not limited by the illustrated configuration. For example, the PET layer 204 may be disposed adjacent the thermoplastic interlayer 210. In another example, the disposition of any one of the components may be switched with another component.

In at least one implementation, the laminates 100, 200 and/or thermoplastic interlayers 106, 206, 210 may be utilized in the preparation or fabrication of a multilayer laminate or multilayer laminate assembly, such as a laminated glazing unit, an insulated glass unit (IGU), a window, a windshield, or the like. For example, the laminate 100, 200 and/or thermoplastic interlayers 106, 206, 210 may be utilized in the preparation or fabrication of windows for vehicles and buildings, impact resistance devices, such as bulletproof glass, decorative films for windows, walls or doors, window tinting, colored or mirrored glass, window films, or the like.

In an exemplary embodiment, the optical film includes one or more thermoplastic polyurethane resins, a first UV absorber and a second UV absorber. The first UV absorber is selected from the group consisting of the benzotriazole UV absorbers and triazine UV absorbers and the second UV absorber is selected from the group consisting of imidazole UV absorbers and cyanoacetate UV absorbers.

The second UV absorber may include one or more imidazole UV absorbers selected from the group consisting of (2,3-Dimethylphenyl) (1H-imidazol-4-yl) methanone, (2,3-Dimethylphenyl) (1H-imidazol-5-yl) methanone, 4-(2′,3′-Dimethylbenzoyl) imidazole, (2,3-Dimethylphenyl) (1-(triphenylmethyl)-1H-imidazol-4-yl) methanone, (2,3-dimethylphenyl) (1-trityl-1 h-imidazol-4-yl) methanone, (2,3-Dimethylphenyl) [1-(triphenylmethyl)-1H-imidazol-4-yl]methanone, derivatives thereof, salts thereof, and compounds thereof. In an exemplary embodiment, the second UV absorber includes methoxypropylamino cyclohexenylidene ethoxyethylcyanoacetate.

In certain embodiments, the optical film includes a light stabilizer.

In certain embodiments, the optical film blocks at least 99% of light having a wavelength ranging from about 380 nm to about 400 nm.

In another exemplary embodiment, a UV blocking film includes a layer of thermoplastic polyurethane and a UV blocker disposed in or coated on the thermoplastic polyurethane layer. The UV blocker is selected from the group consisting of imidazole UV absorbers and cyanoacetate UV absorbers. The UV blocking film blocks greater than or equal to about 95% of light having a wavelength of from about 380 nm to about 400 nm.

In certain embodiments, the UV blocker is selected from the group consisting of (2,3-Dimethylphenyl) (1H-imidazol-4-yl) methanone) and (2,3-Dimethylphenyl) [1-(trityl)-1H-imidazol-4-yl]methanone. In an exemplary embodiment, the UV blocker includes methoxypropylamino cyclohexenylidene ethoxyethylcyanoacetate.

The UV blocker may be present in an amount of about 0.1 wt % to about 2.0 wt % of a total weight percentage of the thermoplastic polyurethane. The UV blocker may a a thickness of from about 0.002 to about 0.025 inches.

In another exemplary embodiment, a laminate includes a first optically transparent sheet, a second optically transparent sheet and an interlayer interposed between the first and second optically transparent sheets. The interlayer includes a thermoplastic polyurethane and a UV blocker disposed in the thermoplastic polyurethane layer. The UV blocker is selected from the group consisting of imidazole UV absorbers and cyanoacetate UV absorbers.

In certain embodiments, the UV blocker is selected from the group consisting of (2,3-Dimethylphenyl) (1H-imidazol-4-yl) methanone), (2,3-Dimethylphenyl) [1-(trityl)-1H-imidazol-4-yl]methanone, and methoxypropylamino cyclohexenylidene ethoxyethylcyanoacetate.

In certain embodiments, the laminate further includes an infrared (IR) cutoff layer interposed between the first and second optically transparent sheets.

In certain embodiments, the laminate further includes one or more of: 1) a transparent electromagnetic interference (EMIM) shielding in, or on, the IR cutoff layer; or 2) a transparent antenna in, or on, the IR cutoff layer.

In certain embodiments, the laminate further includes a low emissivity layer interposed between the first and second optically transparent sheets.

Methods for preparing or fabricating the laminates 100, 200 are described. The method may include preparing a thermoplastic composition including the one or more thermoplastic polymers and the functional elements (e.g., UV absorbers, light stabilizers, etc.). In at least one implementation, the thermoplastic composition may be prepared by combining, mixing, or otherwise contacting the thermoplastic polymers with the functional elements via a screw cast film extruder via dry blending. For example, the method may include combining the thermoplastic polymers, the UV absorbers, and the light stabilizers with one another in a single or twin screw extruder to prepare the thermoplastic composition. The thermoplastic composition may be directed through a die and extruded as a flat film or layer.

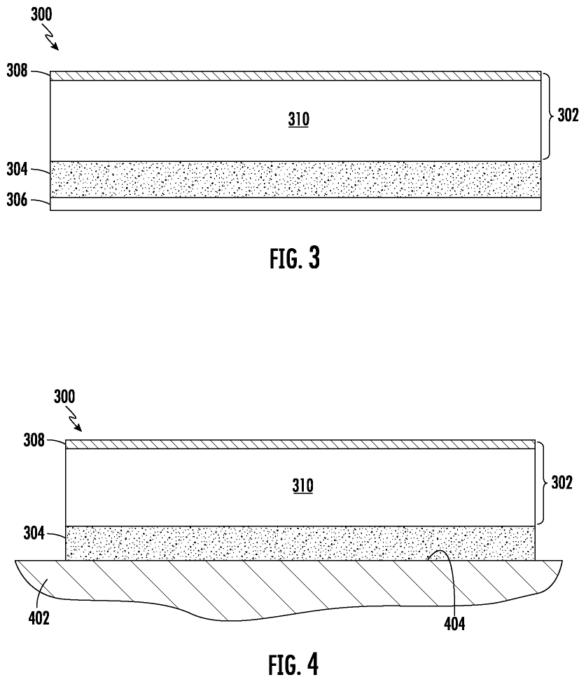

FIG. 3 illustrates a schematic cross-sectional view of an exemplary polyurethane-based bandage 300. The bandage 300 may include any suitable bandage or dressing, such as an elastic or adhesive bandage, dressing, wound dressing, compression bandage, surgical dressing, Tulle dressing, alginate dressing, foam dressing, hydrogel dressing, hydrocolloid dressing, gauze roll, gauze pad and the like. The polyurethane-based bandage 300 may include one or more of a backing layer 302, a therapeutic layer 304, a release liner 306, or any combination thereof. For example, as illustrated in FIG. 3, the polyurethane-based bandage 300 may include the backing layer 302, the therapeutic layer 304 disposed adjacent the backing layer 302, and the release liner 306 disposed adjacent the therapeutic layer 304. It should be appreciated that the polyurethane-based bandage 300 may include additional layers (not shown) or a plurality of any one or more of the backing layers 302, the therapeutic layer 304, and the release liner 306. In at least one implementation, the backing layer 302 may be an outermost layer of the polyurethane-based bandage 300. In another implementation, the polyurethane-based bandage 300 may omit the backing layer 302 and the therapeutic layer 304 may be the outermost layer of the polyurethane-based bandage 300. The backing layer 302 may be flexible, water-resistant or water impermeable, breathable, or a combination thereof. The backing layer 302 may be capable of or configured to protect the wound or skin surface from external contamination and prevent water or bacteria from contacting the wound or skin surface. The backing layer 302 may also be capable of or configured to allow for the exchange of air and moisture vapor to facilitate wound healing.

In at least one embodiment, the backing layer 302 may include a substrate 308, a support layer or film 310, or a combination thereof. For example, as illustrated in FIG. 3, the backing layer 302 may include the substrate 308 and the support layer 310 disposed on or adjacent the substrate 308. The substrate 308 may be any substrate capable of or configured to support the support layer 310. For example, the substrate 308 may be a paper-based carrier substrate, a polymer-based carrier substrate, a thermoplastic-based carrier substrate, or a combination thereof. The substrate 308 may be transparent or semi-transparent. While FIG. 3 illustrates the backing layer 302 as having the substrate 308 and the support layer 310, it should be appreciated that the backing layer 302 may only include the support layer 310. For example, the substrate 308 may be utilized in the manufacture of the backing layer 302 and subsequently removed, leaving only the support layer 310. Accordingly, the substrate 308 may be utilized to facilitate the manufacture of the backing layer (e.g., via melt extrusion casting), but may be excluded from the backing layer 302 of the polyurethane-based wound dressing 300.

Support layer 310 and/or substrate 308 include a layer of thermoplastic polyurethane and a UV blocker disposed in the thermoplastic polyurethane layer. The UV blocker includes a material in the imidazole family, a material in the cyanoacetate family or a combination thereof. As discussed above, the UV blockers in the layer block greater than or equal to about 98% of light having wavelengths of about 380 nm to about 400 nm, while mitigating certain risks, such as carcinogenic, mutagenic and reproductive toxicity risks, that could otherwise raise environmental or health concerns. In embodiments, the UV blocker(s) in the structure block greater than or equal to about 99%, or greater than or equal to about 99.9% of light having wavelengths of about 380 nm to about 400 nm. In certain embodiments, the combination of UV blockers in the structure blocker a substantial portion of light having wavelengths above 400 nm, such as up to about 410 nm or up to about 420 nm.

In an exemplary operation or use of the polyurethane-based bandage 300, with continued reference to FIGS. 3 and 4, the release liner 306 may be separated from the therapeutic layer 304 to expose the adhesive surface of the therapeutic layer 304, as illustrated in FIG. 4. The polyurethane-based bandage 300 may be disposed on or adjacent the site of treatment, which may be the skin 402 of the user/patient, as illustrated in FIG. 4. As further illustrated in FIG. 4, the polyurethane-based bandage 300 may be disposed on or adjacent the skin 402 such that the therapeutic layer 304 contacts a surface 404 of the skin 402 and adheres thereto.

The UV blockers block the transmission of UV light to the patient's skin. UV light can impair the body's natural healing processes and weaken the skin's defense mechanisms, making it easier for bacteria and other pathogens to enter a wound or other skin disorder. UV exposure can also cause hyperpigmentation (darkening) of the skin, leading to uneven skin tone around the target site, such as a wound or disorder. UV light can also directly damage skin cells, particularly the DNA in those cells and can suppress the immune system in the area of a wound. In addition, prolonged exposure to UV light, especially on compromised skin such as a wound site, can increase the risk of developing skin cancer.

In an exemplary embodiment, a bandages includes a layer of thermoplastic polyurethane and a UV blocker disposed in the thermoplastic polyurethane layer. The UV blocker is selected from the group consisting of imidazole UV absorbers and cyanoacetate UV absorbers.

In certain embodiments, the bandage further includes a therapeutic layer in contact with the layer of thermoplastic polyurethane.

In certain embodiments, the bandage further includes a release liner adjacent the therapeutic layer.

In certain embodiments, the UV blocker is selected from the group consisting of (2,3-Dimethylphenyl) (1H-imidazol-4-yl) methanone), (2,3-Dimethylphenyl) [1-(trityl)-1H-imidazol-4-yl]methanone, and methoxypropylamino cyclohexenylidene ethoxyethylcyanoacetate.

In certain embodiments, the UV blocker is present in about 0.1 wt % to about 2.0 wt % of a total weight percentage of the thermoplastic polyurethane layer.

### EXAMPLES

The examples and other implementations described herein are exemplary and not intended to be limiting in describing the full scope of compositions and methods described herein. Equivalent changes, modifications, and variations of specific implementations, materials, compositions, and methods may be made within the scope of the implementations or embodiments described herein, with substantially similar results.′

### Example 1

Comparative laminates (C1)-(C5) were prepared by disposing a thermoplastic interlayer between two rigid layers/plies, namely, two panes of 0.125 in borosilicate glass. The thermoplastic interlayers were prepared according to Table 1. Specifically, comparative laminate (C1) included a thermoplastic interlayer prepared from 0.7 wt % of a UV absorber of the benzotriazole family and a thermoplastic polyether-based polyurethane resin. Comparative laminate (C2) included a thermoplastic interlayer prepared from 1.0 wt % of the UV absorber of the benzotriazole family and the thermoplastic polyether-based polyurethane resin. Comparative laminate (C3) excluded the UV absorber. Comparative laminate (C4) was prepared by extruding a thermoplastic polyurethane resin having a thickness of about 0.002 in without a UV absorber. Comparative laminate (C5) was prepared by extruding a thermoplastic polyurethane resin having a thickness of about 0.002 in with a UV absorber of the benzotriazole family.

Exemplary laminates (E1)-(E6) were prepared by combining the components according to Table 1. It should be appreciated that the exemplary laminates (E1)-(E6) utilized UV absorbers of the imidazole family or the cyanoacetate family. Exemplary laminates (E1)-(E3) were prepared by combining the respective UV absorbers and the respective TPU via melt blending. Exemplary laminates (E4-E6), however, was prepared by extruding the TPU as a film, and coating the TPU film with the UV absorber. Note that laminate (E4) included a UV absorber of the cyanoacetate family (Anthelios UVMune 400 sunscreen, commercially available from La Roche Posay of New York, NY), while laminates (E5) and (E6) included a different UV absorber of the cyanoacetate family (Methoxypropylamino cyclohexenylidene ethoxyethylcyanoacetate (IUPAC: 2-ethoxyethyl(2Z)-2-cyano-2-[3-(3-methoxypropylamino)cyclohex-2-en-1-ylidene]acetate); CAS No. 1419401-88-9).

Exemplary laminates (E3)-(E6) were not laminated between two panes of borosilicate glass. Each of the comparative laminates (C1)-(C5) and the exemplary laminates (E1)-(E6) were evaluated for yellowing index, haze, light transmission, color (L, a*, b*), and/or light transmission at varying wavelengths. The results are summarized in Table 2.

TABLE 1

| | | | | | Imidazole | Imidazole | | |
|---|---|---|---|---|---|---|---|---|
| | | | Benzotriazole | Interlayer | UV | UV | Cyanoacetate | |
| | TPU¹ | TPU⁵ | UV Agent² | Thickness | Agent³ | Agent⁴ | UV Agent^{6, 7} | |
| # | (wt %) | (wt %) | (wt %) | (in) | (wt %) | (wt %) | (wt %) | Laminated |
| C1 | 99.30 | — | 0.7 | 0.025 | — | — | — | Y |
| C2 | 99.0 | — | 1.0 | 0.025 | — | — | — | Y |
| C3 | 100 | — | — | 0.015 | — | — | — | Y |
| C4 | — | 100 | — | 0.002 | — | — | — | N |
| C5 | — | 99.30 | 0.7 | 0.002 | — | — | — | N |
| E1 | 99.75 | — | — | 0.015 | 0.25 | — | — | Y |
| E2 | 99.75 | — | — | 0.015 | — | 0.25 | — | Y |
| E3 | — | 99 | — | 0.002 | — | — | 1⁶ | N |
| E4 | — | 99 | — | 0.002 | — | — | 1⁶ | N |
| E5 | — | 99.5 | — | 0.005 | — | — | 0.5⁷ | N |
| E6 | — | 98 | — | 0.005 | — | — | 2⁷ | N |

¹Thermoplastic polyether polyurethane resin; ELASTOLLAN ® L 1275 A 10, commercially available from BASF of Ludwigshafen, Germany
²Phenol, 2-(5-chloro-2H-benzotriazol-2-yl)-6-(1,1-dimethylethyl)-4-methyl; TINUVIN ® 326, commercially available from BASF of Ludwigshafen, Germany; CAS No.: 3896-11-5
³(2,3-Dimethylphenyl) (1H-imidazol-4-yl) methanone); CAS No.: 91874-85-0
⁴(2,3-Dimethylphenyl) [1-(trityl)-1H-imidazol-4-yl] methanone; CAS No. 176721-02-1
⁵Thermoplastic polyurethane resin; ELASTOLLAN ® 1385 A 12, commercially available from BASF of Ludwigshafen, Germany
⁶Anthelios UVMune 400 sunscreen, commercially available from La Roche Posay of New York, NY;
⁷Methoxypropylamino cyclohexenylidene ethoxyethylcyanoacetate (IUPAC: 2-ethoxyethyl (2Z)-2-cyano-2-[3-(3-methoxypropylamino) cyclohex-2-en-1-ylidene] acetate); CAS No. 1419401-88-9

TABLE 2

| | | | | | | | LT@ | LT@ | LT@ | LT@ | |
|---|---|---|---|---|---|---|---|---|---|---|---|
| | Light | | | | | | 380 | 400 | 420 | 440 | UV |
| | Transmission | | | | | | nm | nm | nm | nm | Cutoff |
| # | (%) | Haze | YI | L | a* | b* | (%) | (%) | (%) | (%) | (nm) |
| C1 | 94.4 | 0.68 | 2.7 | 97.3 | −1.16 | 2.33 | — | 0.229 | — | — | 401 |
| C2 | 94.5 | 0.59 | 3.45 | 97.3 | −1.16 | 2.33 | — | 0.229 | — | — | 401 |
| C3 | — | — | — | — | — | — | 1.47 | 74.49 | 89.01 | 90.27 | — |
| C4 | — | — | — | — | — | — | 66.42 | 69.43 | 71.25 | 73.14 | — |
| C5 | — | — | — | — | — | — | 0.33 | 25.31 | 63.47 | 69.23 | — |
| E1 | — | — | — | — | — | — | 0.03 | 0.33 | 33.73 | 82.54 | — |
| E2 | — | — | — | — | — | — | −0.01 | 0.02 | 64.66 | 89.76 | — |
| E3 | — | — | — | — | — | — | 9.37 | 37.66 | 54.59 | 58.89 | — |
| E4 | — | — | — | — | — | — | 0.02 | 2.56 | 59.49 | 66.39 | — |
| E5 | 86.7 | 63.6 | 21.63 | 83.76 | −2.03 | 13.47 | −0.043 | −0.036 | 33.37 | 49.48 | 400 |
| E6 | 87.8 | 60.8 | 22.24 | 86.98 | −3.56 | 14.34 | −0.05 | −0.055 | 27.2 | 55.66 | 410 |

As indicated in Table 2, comparative laminate (C1), which included about 0.7 wt % of the UV absorber, blocked greater than 99% of the transmission spectrum at wavelengths below about 398 nm As further indicated in Table 2, increasing the amount of the UV absorber to about 1 wt %, as in comparative laminate (C2), resulted in greater than 99% of the transmission spectrum being blocked at wavelengths below about 401 nm. In both comparative laminate (C1) and (C2), a substantial portion of UVA electromagnetic radiation or light was blocked. As further indicated in Table 2, the comparative laminate (C3), which excluded the UV absorber exhibited light transmission values at about 380 nm, about 400 nm, about 420 nm, and about 440 nm of about 1.46%, about 74.49%, about 89.01%, and 90.27%, respectively.

As indicated in Table 2, exemplary laminate (E1) exhibited light transmission values at about 380 nm, about 400 nm, about 420 nm, and about 440 nm of about 0.03%, about 0.33%, about 33.73% and about 82.54%, respectively. Accordingly, the exemplary laminate (E1), which included the UV absorber of the imidazole family exhibited a surprising and unexpected increase in blocking UVA over comparative laminate (C3) at wavelengths up to about 400 nm, and further exhibited a surprising and unexpected increase in blocking blue visible light at wavelengths up to about 420 nm.

Exemplary laminate (E2), which included the UV absorber of the imidazole family, namely, (2,3-Dimethylphenyl) [1-(trityl)-1H-imidazol-4-yl]methanone, exhibited light transmission values at about 380 nm, about 400 nm, about 420 nm, and about 440 nm of about-0.01%, about 0.02%, about 64.66% and about 89.76%, respectively. Exemplary laminate (E2) demonstrated a surprising and unexpected increase in blocking UVA as compared to comparative laminate (C3) at wavelengths up to about 400 nm, and further significant improvement in blocking blue visible light at wavelengths up to about 420 nm.

Exemplary laminate (E3), which included the UV absorber of the cyanoacetate family, exhibited light transmission values at about 380 nm, about 400 nm, about 420 nm, and about 440 nm of about 9.37%, about 37.66%, about 54.59% and about 58.89%, respectively. Exemplary laminate (E3) demonstrated a surprising and unexpected increase in blocking UV as compared to comparative laminate (C4) at wavelengths up to about 400 nm. Exemplary laminate (E3), however, exhibited relatively lower blocking of UV electromagnetic radiation as compared to comparative laminate (C5).

Exemplary laminate (E4), which included a coating of the UV absorber of the cyanoacetate family, exhibited light transmission values at about 380 nm, about 400 nm, about 420 nm, and about 440 nm of about 0.02%, about 2.56%, about 59.49%, and about 66.39%, respectively. Exemplary laminate (E4) demonstrated a surprising and unexpected increase in blocking UVA as compared to comparative laminate (C4) at wavelengths up to about 400 nm.

Exemplary laminate (E5), which included the UV absorber of the cyanoacetate family, exhibited light transmission values at about 380 nm, about 400 nm, about 420 nm, and about 440 nm of about-0.043%, about-0.036%, about 33.37% and about 49.48%, respectively. Exemplary laminate (E5) demonstrated a surprising and unexpected increase in blocking UV as compared to comparative laminate (C4) at wavelengths up to about 400 nm and blue light further up to 440 nm while maintaining total visible light transmission (VLT) above 85%. Exemplary laminate (E5) also exhibited an increase in UV blocking as compared to comparative laminate (C5). UV Cutoff, as defined by the wavelength where light transmission falls below 1%, was somewhere between 400 and 410 nm.

Exemplary laminate (E6), which included the UV absorber of the cyanoacetate family, exhibited light transmission values at about 380 nm, about 400 nm, about 420 nm, and about 440 nm of about-0.05%, about-0.055%, about 27.2% and about 55.66%, respectively. Exemplary laminate (E6) demonstrated a surprising and unexpected increase in blocking UV as compared to comparative laminate (C4) at wavelengths up to about 400 nm and blue light further up to 440 nm while maintaining total visible light transmission (VLT) above 85%. Exemplary laminate (E6) also exhibited an increase in UV blocking as compared to comparative laminate (C5). UV Cutoff, as defined by the wavelength where light transmission falls below 1%, was somewhere between 410 and 420 nm, and higher as compared to exemplary laminate (E5)

As indicated in Table 2, comparative laminate (C5), which included about 0.7 wt % of the UV absorber from the benzotriazole family exhibited a significant improvement in blocking UVA over comparative example (C4) at wavelengths up to about 400 nm.

The foregoing demonstrates that utilizing one or more of the UV absorbers of the imidazole family and/or the cyanoacetate family in a thermoplastic interlayer provides both surprising and unexpected blocking of UV electromagnetic radiation at wavelengths up to about 400 nm. The results are synergistic when the UV absorber of the imidazole family and/or the cyanoacetate family are combined with one another, or when combined with one or more UV absorbers from the benzotriazole family, the triazine family, the benzophenone family, the benzylidene malonate family, or any combination thereof.

In particular, it should be noted that the cyanoacetate UV agent was particularly successful at blocking UV light in a thermoplastic layer of about 2 mils thickness. Thus, this UV agent may be particularly useful in applications that require thicknesses in this range, such as bandages and wound dressings. The imidazole agent was particularly useful in a thermoplastic layer of about 15 mils in thickness. Thus, this UV agent may be useful in applications that require thicknesses in this range, such as laminates for windows and the like.

While the devices, systems, and methods have been described in detail herein in accordance with certain preferred implementations thereof, many modifications and changes therein may be affected by those skilled in the art. Accordingly, the foregoing description should not be construed to be limited thereby but should be construed to include such aforementioned obvious variations and be limited only by the spirit and scope of the following claims.

For example, in a first aspect, a first embodiment a structure including a layer of thermoplastic polyurethane and a UV blocker disposed in the thermoplastic polyurethane layer, the UV blocker including a material in the imidazole family, a material in the cyanoacetate family or a combination thereof.

A second embodiment is the first embodiment, wherein the UV blocker includes a material in the imidazole family.

A third embodiment is any combination of the above embodiments, wherein the UV blocker includes a material in the cyanoacetate family.

A 4^{th} embodiment is any combination of the above embodiments, wherein the UV blocker includes a first UV blocker including a material in cyanoacetate family and a second UV blocker including a material in the imidazole family.

A 5^{th} embodiment is any combination of the above embodiments, wherein the material in the imidazole family is selected from the group consisting of (2,3-Dimethylphenyl) (1H-imidazol-4-yl) methanone, (2,3-Dimethylphenyl) (1H-imidazol-5-yl) methanone, 4-(2′,3′-Dimethylbenzoyl) imidazole, (2,3-Dimethylphenyl) (1-(triphenylmethyl)-1H-imidazol-4-yl) methanone, (2,3-dimethylphenyl) (1-trityl-1 h-imidazol-4-yl) methanone, (2,3-Dimethylphenyl) [1-(triphenylmethyl)-1H-imidazol-4-yl]methanone, derivatives thereof, salts thereof, compounds thereof, or any combination thereof.

A 6^{th} embodiment is any combination of the above embodiments, wherein the material in the imidazole family is selected from the group consisting of (2,3-Dimethylphenyl) (1H-imidazol-4-yl) methanone, (2,3-Dimethylphenyl) (1-(triphenylmethyl)-1H-imidazol-4-yl) methanone, or any combination thereof.

A 7^{th} embodiment is any combination of the above embodiments, wherein the material in the imidazole family includes (2,3-Dimethylphenyl) (1H-imidazol-4-yl) methanone.

An 8^{th} embodiment is any combination of the above embodiments, wherein the material in the imidazole family includes (2,3-Dimethylphenyl) (1-(triphenylmethyl)-1H-imidazol-4-yl) methanone.

A 9^{th} embodiment is any combination of the above embodiments, wherein the material in the cyanoacetate family is represented by the formula:

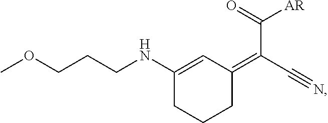

wherein A is —O— or —NH—, wherein R is a C1-C22 alkyl group, a C2-C22 alkenyl group, a C2-C22 alkynyl group, a C3-C22 cycloalkyl group, or a C3-C22 cycloalkenyl group, and wherein each of the groups is interrupted by one or more oxygens (—O—).

A 10^{th} embodiment is any combination of the above embodiments, wherein the material in the cyanoacetate family is represented by the formula:

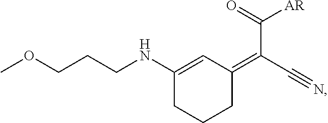

wherein A is —O— or —NH—, wherein R is a C1-C22 alkyl group, a C2-C22 alkenyl group, a C2-C22 alkynyl group, a C3-C22 cycloalkyl group, or a C3-C22 cycloalkenyl group, and wherein each of the groups is not interrupted by one or more oxygens (—O—).

An 11^{th} embodiment is any combination of the above embodiments, wherein the material in the cyanoacetate family includes methoxypropylamino cyclohexenylidene ethoxyethylcyanoacetate (MCE).

A 12^{th} embodiment is any combination of the above embodiments, wherein a weight ratio of the first UV blocker to the second UV blocker is from about 10:1 to about 1:10.

A 13^{th} embodiment is any combination of the above embodiments, further including a second UV blocker including a material in benzotriazole family, the triazine family, or a combination thereof.

A 14^{th} embodiment is any combination of the above embodiments, wherein the second UV blocker includes a material in the triazine family.

A 15^{th} embodiment is any combination of the above embodiments, wherein the second UV blocker includes a material in in the benzotriazole family.

A 16^{th} embodiment is any combination of the above embodiments, wherein the second UV blocker includes 2-(4,6-diphenyl-1,3,5-triazin-2-yl)-5-[(hexyl)oxy]-phenol.

A 17^{th} embodiment is any combination of the above embodiments, wherein the second UV blocker is represented by the formula:

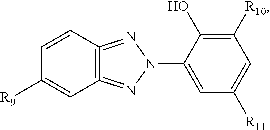

wherein each of R₉, R₁₀, and R₁₁ are independently a hydrogen, a halogen, or a group represented by CₐH_{b}N_{c}O_{d}Sₑ, where each of a, b, c, d, and e may independently be from 0 to 30.

An 18^{th} embodiment is any combination of the above embodiments, wherein the second UV blocker includes 2-(2H-benzotriazol-2-yl)-4,6-bis(1,1-dimethylpropyl)-phenol.

A 19^{th} embodiment is any combination of the above embodiments, further including a light stabilizer.

A 20^{th} embodiment is any combination of the above embodiments, wherein the light stabilizer is a hindered amine light stabilizer (HALS).

A 21^{st} embodiment is any combination of the above embodiments, wherein the light stabilizer is the reaction product of bis(1,2,2,6,6-pentamethyl-4-piperidyl) sebacate and methyl 1,2,2,6,6-pentamethyl-4-piperidyl sebacate.

A 22^{nd} embodiment is any combination of the above embodiments, wherein the structure blocks greater than or equal to about 95% of light having a wavelength of from about 380 nm to about 400 nm.

A 23^{rd} embodiment is any combination of the above embodiments, wherein the structure blocks greater than or equal to about 98% of light having a wavelength of from about 380 nm to about 400 nm.

A 24^{th} embodiment is any combination of the above embodiments, wherein the structure blocks greater than or equal to about 99% of light having a wavelength of from about 380 nm to about 400 nm.

A 25^{th} embodiment is any combination of the above embodiments, wherein the structure blocks greater than or equal to about 98% of light having a wavelength of about 400 nm.

A 26^{th} embodiment is any combination of the above embodiments, wherein the structure blocks greater than or equal to about 99% of light having a wavelength of about 400 nm.

A 27^{th} embodiment is any combination of the above embodiments, wherein the structure blocks greater than or equal to about 99.9% of light having a wavelength of about 400 nm.

A 28^{th} embodiment is any combination of the above embodiments, wherein the structure has a thickness of from about 10 Mils to about 60 Mils.

A 29^{th} embodiment is any combination of the above embodiments, wherein the structure has a yellowness index, as determined according to reference test ASTM D1925, of less than or equal to about 3%.

A 30^{th} embodiment is any combination of the above embodiments, wherein the structure has a visible light transmission, as measured according to reference test ASTM D1003, of greater than or equal to about 90%.

A 31^{st} embodiment is any combination of the above embodiments, wherein the structure is substantially free of UV blockers identified by the Registration, Evaluation, Authorization, and Restriction of Chemicals (REACH).

A 32^{nd} embodiment is a laminate, including a first optically transparent sheet; a second optically transparent sheet; and an interlayer interposed between the first and second optically transparent outer sheets, wherein the interlayer includes a thermoplastic polyurethane and a UV blocker disposed in the thermoplastic polyurethane layer, the UV blocker including a material in the imidazole family, a material in the cyanoacetate family or a combination thereof.

A 33^{rd} embodiment is any combination of the above embodiments, wherein the UV blocker includes a material in the imidazole family.

A 34^{th} embodiment is any combination of the above embodiments, wherein the UV blocker includes a material in the cyanoacetate family.

A 35^{th} embodiment is any combination of the above embodiments, wherein the UV blocker includes a first UV blocker including a material in cyanoacetate family and a second UV blocker including a material in the imidazole family.

A 36^{th} embodiment is any combination of the above embodiments, further including an infrared (IR) cutoff layer interposed between the first and second optically transparent sheets.

A 37^{th} embodiment is any combination of the above embodiments, further including a transparent electromagnetic interference (EMIM) shielding in, or on, the IR cutoff layer.

A 38^{th} embodiment is any combination of the above embodiments, further including a transparent antenna in, or on, the IR cutoff layer.

A 39^{th} embodiment is any combination of the above embodiments, further including a low emissivity layer interposed between the first and second optically transparent sheets.

A 40^{th} embodiment is any combination of the above embodiments, wherein the UV blocker includes more of (2,3-Dimethylphenyl) (1H-imidazol-4-yl) methanone, (2,3-Dimethylphenyl) (1H-imidazol-5-yl) methanone, 4-(2′,3′-Dimethylbenzoyl) imidazole, (2,3-Dimethylphenyl) (1-(triphenylmethyl)-1H-imidazol-4-yl) methanone, (2,3-dimethylphenyl) (1-trityl-1 h-imidazol-4-yl) methanone, (2,3-Dimethylphenyl) [1-(triphenylmethyl)-1H-imidazol-4-yl]methanone, derivatives thereof, salts thereof, compounds thereof, or any combination thereof.

A 41^{st} embodiment is any combination of the above embodiments, wherein the second UV blocker is represented by the formula:

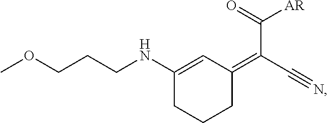

wherein A is —O— or —NH—, wherein R is a C1-C22 alkyl group, a C2-C22 alkenyl group, a C2-C22 alkynyl group, a C3-C22 cycloalkyl group, or a C3-C22 cycloalkenyl group, and wherein each of the groups is interrupted by one or more oxygens (—O—).

A 42nd embodiment is any combination of the above embodiments, wherein the second UV blocker is represented by the formula:

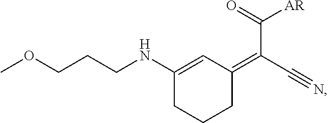

wherein A is —O— or —NH—, wherein R is a C1-C22 alkyl group, a C2-C22 alkenyl group, a C2-C22 alkynyl group, a C3-C22 cycloalkyl group, or a C3-C22 cycloalkenyl group, and wherein each of the groups is not interrupted by one or more oxygens (—O—).

A 43^{rd} embodiment is any combination of the above embodiments, wherein the second UV blocker includes methoxypropylamino cyclohexenylidene ethoxyethylcyanoacetate (MCE).

A 44^{th} embodiment is any combination of the above embodiments, wherein a weight ratio of the first UV blocker to the second UV blocker is from about 10:1 to about 1:10.

A 45^{th} embodiment is any combination of the above embodiments, further including a second UV blocker including a material in the benzotriazole family, the triazine family, or a combination thereof.

A 46^{th} embodiment is any combination of the above embodiments, wherein the third UV blocker includes 2-(4,6-diphenyl-1,3,5-triazin-2-yl)-5-[(hexyl)oxy]-phenol.

A 47^{th} embodiment is any combination of the above embodiments, wherein the third UV blocker is represented by the formula:

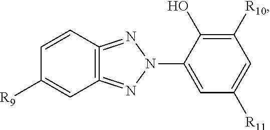

wherein each of R₉, R₁₀, and R₁₁ are independently a hydrogen, a halogen, or a group represented by CₐH_{b}N_{c}O_{d}Sₑ, where each of a, b, c, d, and e may independently be from 0 to 30.

A 48^{th} embodiment is any combination of the above embodiments, wherein the structure blocks greater than or equal to about 98% of light having a wavelength of about 400 nm.

A 49^{th} embodiment is any combination of the above embodiments, wherein the structure blocks greater than or equal to about 99% of light having a wavelength of about 400 nm.

A 50^{th} embodiment is any combination of the above embodiments, wherein the structure blocks greater than or equal to about 99.9% of light having a wavelength of about 400 nm.

A 51^{st} embodiment is a window and any combination of the above embodiments.

A 52^{nd} embodiment is a bandage including a layer of thermoplastic polyurethane and a UV blocker disposed in the thermoplastic polyurethane layer, the UV blocker including a material in the imidazole family, a material in the cyanoacetate family or a combination thereof.

A 53^{rd} embodiment is the 52^{nd} embodiment, further including a therapeutic layer in contact with the layer of thermoplastic polyurethane.

A 54^{th} embodiment is any combination of the above embodiments, further including a release liner adjacent the therapeutic layer.

A 55^{th} embodiment is any combination of the above embodiments, wherein the UV blocker includes a material in the imidazole family.

A 60^{th} embodiment is any combination of the above embodiments, wherein the UV blocker includes a material in the cyanoacetate family.

A 61^{st} embodiment is any combination of the above embodiments, wherein the UV blocker includes a first UV blocker including a material in cyanoacetate family and a second UV blocker including a material in the imidazole family.

A 62^{nd} embodiment is any combination of the above embodiments, wherein the layer of thermoplastic polyurethane has a thickness of about 1 mil to about 3 mils.

A 63^{rd} embodiment is any combination of the above embodiments, wherein the material in the imidazole family is selected from the group consisting of (2,3-Dimethylphenyl) (1H-imidazol-4-yl) methanone, (2,3-Dimethylphenyl) (1H-imidazol-5-yl) methanone, 4-(2′,3′-Dimethylbenzoyl) imidazole, (2,3-Dimethylphenyl) (1-(triphenylmethyl)-1H-imidazol-4-yl) methanone, (2,3-dimethylphenyl) (1-trityl-1 h-imidazol-4-yl) methanone, (2,3-Dimethylphenyl) [1-(triphenylmethyl)-1H-imidazol-4-yl]methanone, derivatives thereof, salts thereof, compounds thereof, or any combination thereof.

A 64^{th} embodiment is any combination of the above embodiments, wherein the material in the imidazole family is selected from the group consisting of (2,3-Dimethylphenyl) (1H-imidazol-4-yl) methanone, (2,3-Dimethylphenyl) (1-(triphenylmethyl)-1H-imidazol-4-yl) methanone, or any combination thereof.

A 65^{th} embodiment is any combination of the above embodiments, wherein the material in the imidazole family includes (2,3-Dimethylphenyl) (1H-imidazol-4-yl) methanone.

A 66^{th} embodiment is any combination of the above embodiments, wherein the material in the imidazole family includes (2,3-Dimethylphenyl) (1-(triphenylmethyl)-1H-imidazol-4-yl) methanone.

A 67^{th} embodiment is any combination of the above embodiments, wherein the material in the cyanoacetate family is represented by the formula:

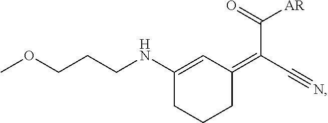

wherein A is —O— or —NH—, wherein R is a C1-C22 alkyl group, a C2-C22 alkenyl group, a C2-C22 alkynyl group, a C3-C22 cycloalkyl group, or a C3-C22 cycloalkenyl group, and wherein each of the groups is interrupted by one or more oxygens (—O—).

A 68^{th} embodiment is any combination of the above embodiments, wherein the material in the cyanoacetate family is represented by the formula:

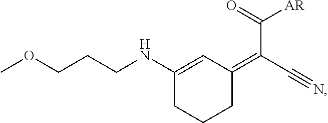

wherein A is —O— or —NH—, wherein R is a C1-C22 alkyl group, a C2-C22 alkenyl group, a C2-C22 alkynyl group, a C3-C22 cycloalkyl group, or a C3-C22 cycloalkenyl group, and wherein each of the groups is not interrupted by one or more oxygens (—O—).

A 69^{th} embodiment is any combination of the above embodiments, wherein the material in the cyanoacetate family includes methoxypropylamino cyclohexenylidene ethoxyethylcyanoacetate (MCE).

A 70^{th} embodiment is any combination of the above embodiments, wherein the UV blocker is present in about 0.1 wt % to about 2.0 wt % of a total weight percentage of the thermoplastic polyurethane layer.

A 71^{st} embodiment is any combination of the above embodiments, wherein the UV blocker present in about 0.2 wt % to about 1.2 wt % of a total weight percentage of the thermoplastic polyurethane layer.

A 72^{nd} embodiment is any combination of the above embodiments, wherein the UV blocker includes a material in the cyanoacetate family in about 0.5 wt % to about 2.0% of a total weight percentage of the thermoplastic polyurethane layer.

A 73^{rd} embodiment is any combination of the above embodiments, wherein the UV blocker includes a material in the imidazole family in about 0.25 wt % of a total weight percentage of the thermoplastic polyurethane layer.

A 74^{th} embodiment is a wound dressing including the bandage of any combination of the above embodiments.

A 75^{th} embodiment is an optical film comprising: one or more thermoplastic polyurethane resins; a first UV absorber, and a second UV absorber, wherein: the first UV absorber is selected from the group consisting of the benzotriazole UV absorbers and triazine UV absorbers, and the second UV absorber is selected from the group consisting of imidazole UV absorbers and cyanoacetate UV absorbers.

A 76^{th} embodiment is the 75^{th} embodiment, wherein the second UV absorber comprises one or more imidazole UV absorbers selected from the group consisting of (2,3-Dimethylphenyl) (1H-imidazol-4-yl) methanone, (2,3-Dimethylphenyl) (1H-imidazol-5-yl) methanone, 4-(2′,3′-Dimethylbenzoyl) imidazole, (2,3-Dimethylphenyl) (1-(triphenylmethyl)-1H-imidazol-4-yl) methanone, (2,3-dimethylphenyl) (1-trityl-1 h-imidazol-4-yl) methanone, (2,3-Dimethylphenyl) [1-(triphenylmethyl)-1H-imidazol-4-yl]methanone, derivatives thereof, salts thereof, and compounds thereof.

A 77^{th} embodiment is any combination of the above embodiments, wherein the second UV absorber comprises methoxypropylamino cyclohexenylidene ethoxyethylcyanoacetate.

A 78^{th} embodiment is any combination of the above embodiments, further comprising a light stabilizer.

A 79^{th} embodiment is any combination of the above embodiments, wherein the optical film blocks at least 99% of light having a wavelength ranging from about 380 nm to about 400 nm.

An 80^{th} embodiment is a UV blocking film comprising: a layer of thermoplastic polyurethane; and a UV blocker disposed in or coated on the thermoplastic polyurethane layer, wherein: the UV blocker is selected from the group consisting of imidazole UV absorbers and cyanoacetate UV absorbers, and the UV blocking film blocks greater than or equal to about 95% of light having a wavelength of from about 380 nm to about 400 nm.

An 81^{st} embodiment is any combination of the above embodiments, wherein the UV blocker is selected from the group consisting of (2,3-Dimethylphenyl) (1H-imidazol-4-yl) methanone) and (2,3-Dimethylphenyl) [1-(trityl)-1H-imidazol-4-yl]methanone.

An 82^{nd} embodiment is any combination of the above embodiments, wherein the UV blocker comprises methoxypropylamino cyclohexenylidene ethoxyethylcyanoacetate.

An 83^{rd} embodiment is any combination of the above embodiments, wherein the UV blocker is present in an amount of about 0.1 wt % to about 2.0 wt % of a total weight percentage of the thermoplastic polyurethane.

An 84^{th} embodiment is any combination of the above embodiments, having a thickness of from about 0.002 to about 0.025 inches.

An 85^{th} embodiment is laminate, comprising: a first optically transparent sheet; a second optically transparent sheet; and an interlayer interposed between the first and second optically transparent sheets, the interlayer comprising: a thermoplastic polyurethane; and a UV blocker disposed in the thermoplastic polyurethane layer, wherein the UV blocker is selected from the group consisting of imidazole UV absorbers and cyanoacetate UV absorbers.

An 86^{th} embodiment is any combination of the above embodiments, wherein the UV blocker is selected from the group consisting of (2,3-Dimethylphenyl) (1H-imidazol-4-yl) methanone), (2,3-Dimethylphenyl) [1-(trityl)-1H-imidazol-4-yl]methanone, and methoxypropylamino cyclohexenylidene ethoxyethylcyanoacetate.

An 87^{th} embodiment is any combination of the above embodiments, further comprising an infrared (IR) cutoff layer interposed between the first and second optically transparent sheets.

An 88^{th} embodiment is any combination of the above embodiments, further comprising one or more of: 1) a transparent electromagnetic interference (EMIM) shielding in, or on, the IR cutoff layer; or 2) a transparent antenna in, or on, the IR cutoff layer.

An 89^{th} embodiment is any combination of the above embodiments, further comprising a low emissivity layer interposed between the first and second optically transparent sheets.

A 90^{th} embodiment is a bandage comprising: a layer of thermoplastic polyurethane; and a UV blocker disposed in the thermoplastic polyurethane layer, wherein the UV blocker is selected from the group consisting of imidazole UV absorbers and cyanoacetate UV absorbers.

A 91^{st} embodiment is any combination of the above embodiments, further comprising a therapeutic layer in contact with the layer of thermoplastic polyurethane.

A 92^{nd} embodiment is any combination of the above embodiments, further comprising a release liner adjacent the therapeutic layer.

A 93^{rd} embodiment is any combination of the above embodiments, wherein the UV blocker is selected from the group consisting of (2,3-Dimethylphenyl) (1H-imidazol-4-yl) methanone), (2,3-Dimethylphenyl) [1-(trityl)-1H-imidazol-4-yl]methanone, and methoxypropylamino cyclohexenylidene ethoxyethylcyanoacetate.

A 94^{th} embodiment is any combination of the above embodiments, wherein the UV blocker is present in about 0.1 wt % to about 2.0 wt % of a total weight percentage of the thermoplastic polyurethane layer.

What is claimed is:

1. An optical film comprising:
one or more thermoplastic polyurethane resins;
a first UV absorber, and
a second UV absorber,
wherein:
the first UV absorber is selected from the group consisting of the benzotriazole UV absorbers and triazine UV absorbers, and
the second UV absorber is selected from the group consisting of imidazole UV absorbers and cyanoacetate UV absorbers.

2. The optical film of claim 1, wherein the second UV absorber comprises one or more imidazole UV absorbers selected from the group consisting of (2,3-Dimethylphenyl) (1H-imidazol-4-yl) methanone, (2,3-Dimethylphenyl) (1H-imidazol-5-yl) methanone, 4-(2′,3′-Dimethylbenzoyl) imidazole, (2,3-Dimethylphenyl) (1-(triphenylmethyl)-1H-imidazol-4-yl) methanone, (2,3-dimethylphenyl) (1-trityl-1h-imidazol-4-yl) methanone, (2,3-Dimethylphenyl) [1-(triphenylmethyl)-1H-imidazol-4-yl]methanone, derivatives thereof, salts thereof, and compounds thereof.

3. The optical film of claim 2, wherein the second UV absorber comprises methoxypropylamino cyclohexenylidene ethoxyethylcyanoacetate.

4. The optical film of claim 1, further comprising a light stabilizer.

5. The optical film of claim 1, wherein the optical film blocks at least 99% of light having a wavelength ranging from about 380 nm to about 400 nm.

6. A UV blocking film comprising:
a layer of thermoplastic polyurethane; and
a UV blocker disposed in or coated on the thermoplastic polyurethane layer, wherein:
the UV blocker is selected from the group consisting of imidazole UV absorbers and cyanoacetate UV absorbers, and
the UV blocking film blocks greater than or equal to about 95% of light having a wavelength of from about 380 nm to about 400 nm.

7. The UV blocking film of claim 6, wherein the UV blocker is selected from the group consisting of (2,3-Dimethylphenyl) (1H-imidazol-4-yl) methanone) and (2,3-Dimethylphenyl) [1-(trityl)-1H-imidazol-4-yl]methanone.

8. The UV blocking film of claim 6, wherein the UV blocker comprises methoxypropylamino cyclohexenylidene ethoxyethylcyanoacetate.

9. The UV blocking film of claim 6, wherein the UV blocker is present in an amount of about 0.1 wt % to about 2.0 wt % of a total weight percentage of the thermoplastic polyurethane.

10. The UV blocking film of claim 6, having a thickness of from about 0.002 to about 0.025 inches.

11. A laminate, comprising:
a first optically transparent sheet;
a second optically transparent sheet; and
an interlayer interposed between the first and second optically transparent sheets, the interlayer comprising:
a thermoplastic polyurethane; and
a UV blocker disposed in the thermoplastic polyurethane layer,
wherein the UV blocker is selected from the group consisting of imidazole UV absorbers and cyanoacetate UV absorbers.

12. The laminate of claim 11, wherein the UV blocker is selected from the group consisting of (2,3-Dimethylphenyl) (1H-imidazol-4-yl) methanone), (2,3-Dimethylphenyl) [1-(trityl)-1H-imidazol-4-yl]methanone, and methoxypropylamino cyclohexenylidene ethoxyethylcyanoacetate.

13. The laminate of claim 11, further comprising an infrared (IR) cutoff layer interposed between the first and second optically transparent sheets.

14. The laminate of claim 12, further comprising one or more of: 1) a transparent electromagnetic interference (EMIM) shielding in, or on, the IR cutoff layer; or 2) a transparent antenna in, or on, the IR cutoff layer.

15. The laminate of claim 11, further comprising a low emissivity layer interposed between the first and second optically transparent sheets.

16. A bandage comprising:
a layer of thermoplastic polyurethane; and
a UV blocker disposed in the thermoplastic polyurethane layer,
wherein the UV blocker is selected from the group consisting of imidazole UV absorbers and cyanoacetate UV absorbers.

17. The bandage of claim 16, further comprising a therapeutic layer in contact with the layer of thermoplastic polyurethane.

18. The bandage of claim 17, further comprising a release liner adjacent the therapeutic layer.

19. The bandage of claim 17, wherein the UV blocker is selected from the group consisting of (2,3-Dimethylphenyl) (1H-imidazol-4-yl) methanone), (2,3-Dimethylphenyl) [1-(trityl)-1H-imidazol-4-yl]methanone, and methoxypropylamino cyclohexenylidene ethoxyethylcyanoacetate.

20. The bandage of claim 17, wherein the UV blocker is present in about 0.1 wt % to about 2.0 wt % of a total weight percentage of the thermoplastic polyurethane layer.
